(19) United States
(12) Patent Application Publication
(10) Pub. No.: US 2019/0351135 A1
(43) Pub. Date: Nov. 21, 2019

(54) TRANSFUSION PUMP

(71) Applicant: E3D AGRICULTURAL COOPERATIVE ASSOCIATION LTD, Merom Hagalil (IL)

(72) Inventors: Ziv NAFTALOVITZ, Merom Hagalil (IL); Ilan SHOPEN, Zefat (IL); Tsachi SHAKED, Merom Hagalil (IL); Ozi BEN ARI, Upper Galilee (IL); Micha SEGEV, Lower Galilee (IL)

(73) Assignee: E3D AGRICULTURAL COOPERATIVE ASSOCIATION LTD, Merom Hagalil (IL)

(21) Appl. No.: 16/478,011

(22) PCT Filed: Jan. 15, 2018
(86) PCT No.: PCT/IL2018/050053

§ 371 Date: Jul. 15, 2019

Related U.S. Application Data
(60) Provisional application No. 62/446,466, filed on Jan. 15, 2017.
(60) Provisional application No. 62/502,786, filed on May 8, 2017.

(51) Int. Cl. A61M 5/142; A61M 5/145

(52) U.S. Cl. CPC A61M 5/14248; A61M 2005/1581; A61M 2005/14204; A61M 5/1452

(57) ABSTRACT

A pump assembly useful for pumping a medicament through an injection port which is removably mountable onto a patient body surface for defining a medicament injection pathway, the pump assembly including a timed pumping assembly adapted for pumping the medicament through the injection port at a given time and a removable mounting adapted for removable mounting of the timed pumping assembly onto the injection port, whereby when the timed pumping assembly is mounted onto the injection port, the medicament can be pumped by the time pumping assembly through the medicament injection pathway.

### REFERENCE TO RELATED APPLICATIONS

Reference is hereby made to U.S. Provisional Patent Application 62/446,466, filed Jan. 15, 2017 and entitled “INFUSION PUMP DEVICE”, and to 62/502,786, filed May 8, 2017 and entitled “H2 Cell Pump Device”, the disclosures of which are incorporated by reference in their entirety and priority of which is hereby claimed pursuant to 37 CFR 1.78(a) (4) and (5)(i).

### FIELD OF THE INVENTION

The present invention relates to medicament transfusion and injection devices generally.

### BACKGROUND OF THE INVENTION

Various types of medicament transfusion and injection devices are known in the art.

### SUMMARY OF THE INVENTION

The present invention seeks to provide an improved transfusion pump and a method of use thereof.

There is thus provided in accordance with an embodiment of the present invention a pump assembly useful for pumping a medicament through an injection port which is removably mountable onto a patient body surface for defining a medicament injection pathway, the pump assembly including: a timed pumping assembly adapted for pumping the medicament through the injection port at a given time; a removable mounting adapted for removable mounting of the timed pumping assembly onto the injection port, whereby when the timed pumping assembly is mounted onto the injection port, the medicament can be pumped by the time pumping assembly through the medicament injection pathway.

Preferably, the pump assembly also includes the injection port. Further preferably, the pumping assembly is removably mountable onto the injection port both prior to and following pumping of at least a portion of the medicament through the injection port.

Still further preferably, the pumping assembly is removable from the injection port and attachable to a different injection port without resulting in undesired pumping of the medicament through the injection port. Yet further preferably, the given time is measured from mounting of the pumping assembly onto the injection port.

In accordance with an embodiment of the present invention, the pumping assembly includes a timer which is actuated by a time duration start switch, the time duration start switch being actuated by mounting of the pumping assembly onto the injection port.

1. Preferably, the given time is a fixed time duration following mounting of the pumping assembly onto the injection port. Further preferably, the pumping assembly includes a top housing portion, and a bottom housing portion fixedly mounted to the top housing portion and defining an interior volume therebetween, which encloses a reservoir assembly extending along a longitudinal axis and a circuit and switch assembly, an underside of said bottom housing portion is at least partially covered by an adhesive layer, which is adapted to be secured to said patient body surface.

Still further preferably, the interior volume includes a pump chamber and a fluid delivery and actuation chamber, which are at least partially separated by a wall portion. Yet further preferably, the timed pumping assembly is adapted to reside within the pump chamber and the removable mounting is adapted to reside within the fluid delivery and actuation chamber.

In accordance with an embodiment of the present invention, the reservoir assembly includes a barrel assembly having an interior volume, a forward closed end and a rearward open end, the barrel assembly also includes a needle hub extending forwardly from the closed end, and a needle, which is partially inserted into the needle hub.

Preferably, the reservoir assembly encloses a pressure generating assembly and a piston displacement controller, which is operative to prevent forward displacement of at least a portion of the pressure generating assembly along the longitudinal axis until a predetermined pressure level is reached within the pressure generating assembly. Further preferably, the removable mounting includes a plurality of mutually concentric arms arranged about an axis, at least one of the arms having a radially inwardly extending snap configured for engagement with the injection port. Still further preferably, the time duration start switch is being mounted within the fluid delivery and actuation chamber and includes a first portion and a second portion, which are axially slidable relative to each other. Yet further preferably, the barrel assembly defines an interior volume having a generally oval cross-section.

Preferably, the barrel assembly having at least one protrusion configured to receive a ratchet assembly therewithin, which communicates with the interior volume of the barrel assembly. Further preferably, the reservoir assembly encloses a first piston assembly, a second piston assembly and a fluid pressure generator enclosed therebetween at least in some of the operative orientations of the pump assembly. Still further preferably, the removable mounting is fixed to the timed pumping assembly. Yet further preferably, the piston displacement controller includes at least a portion of a first piston assembly which is operatively associated with a ratchet assembly when the piston displacement controller is disposed in an actuated operative orientation.

In accordance with another embodiment of the present invention, the piston displacement controller includes an electrical valve incorporated with a needle, which is activated upon receiving of an electrical signal indicating actuation of the fluid pressure generator. Alternatively, the piston displacement controller includes a mechanical valve operatively coupled with a needle, which is activated by pressure exerted thereon following gas discharge from the fluid pressure generator.

Preferably, the reservoir assembly has an at least partially oval cross-section. Further preferably, the ratchet assembly includes a compressible bellow portion and a ratchet tooth fixed thereto and having a straight edge and a tapered edge, the bellow portion is operative to be compressed upon exertion of force on the ratchet assembly. Further preferably, the first piston assembly has a forwardly facing surface and a rearwardly facing surface and the second piston assembly has a forwardly facing surface and a rearwardly facing surface. Still further preferably, the first piston assembly includes at least one ratchet racks, extending longitudinally from a location adjacent to the forwardly facing surface to a location rearwardly disposed with respect to rearwardly facing surface, the at least one ratchet rack includes a plurality of ratchet teeth, each defining a straight edge and a tapered edge. Yet further preferably, the fluid pressure generator has at least one opening for facilitating discharge of gas therefrom following activation of the fluid pressure generator.

In accordance with an embodiment of the present invention, the pressure generating assembly slidably resides within the interior volume of a reservoir assembly. Preferably, the fluid pressure generator is configured to be electrically coupled to the circuit and switch assembly of the pump assembly. Further preferably, the forwardly facing surface of the first piston assembly abuts the closed end of the barrel assembly when the pump assembly is empty of the medicament.

Preferably, the pump assembly also includes a filling port assembly. Further preferably, the filling port assembly includes a filling port housing portion having a mounting portion, the filling port housing portion is configured for enclosing a first septum and a second septum forming a closed chamber therebetween, the first and second septums are sealingly disposed within the filling port housing portion. Still further preferably, the mounting portion of the filling port housing portion is configured to be removably mounted into a fluid delivery and actuation chamber formed within the pumping assembly. Yet further preferably, the reservoir assembly is fixedly supported within the pump chamber, such that axial displacement of the reservoir assembly is prevented.

In accordance with an embodiment of the present invention, the time duration start switch is a proximity sensor. Alternatively, the time duration start switch is a magnet switch. Further alternatively, the time duration start switch is a relay switch.

Preferably, upon mounting of the filling port assembly onto pumping assembly, a needle of the pumping assembly pierces the first septum and extends into the closed chamber. Further preferably, the piston displacement controller is operative both for restraining forward displacement of at least a portion of the pressure generating assembly in certain operative orientations and for fluid tight sealing of gas that is generated by the fluid pressure generator in other operative orientations. Still further preferably, the time duration start switch is activated when the first portion at least partially abuts the second portion. Yet further preferably, the interior volume of the barrel assembly is entirely empty of fluid before filling thereof, thereby obviating the need for priming of the barrel assembly after filling the reservoir assembly with the medicament.

In accordance with an embodiment of the present invention, the rearwardly facing surface of the first piston assembly abuts the forwardly facing surface of the second piston assembly when the fluid pressure generator is disposed in a non-activated operative orientation.

Preferably, a pre-filled syringe is adapted to be operatively coupled to the filling port assembly and a force applied by a user on a portion of the pre-filled syringe causes hydraulic pressure to be applied on the pressure generating assembly and thereby urge rearward sealingly slidable displacement thereof relative to a barrel assembly, such that the interior volume of the barrel assembly is filled with the medicament. Further preferably, the forwardly facing surface of the first piston assembly is rearwardly spaced from the closed end of the barrel assembly when the pump assembly is tilled with the medicament. Still further preferably, at least a portion of the pressure generating assembly has a sealing ring associated therewith. Yet further preferably, the medicament is sealed within the interior volume of the barrel assembly due to fluid-tight sealing engagement of the sealing ring with an inner surface of the barrel assembly.

In accordance with an embodiment of the present invention, forward displacement of the pressure generating assembly is prevented by means of engagement of the tooth of the ratchet assembly with one of the plurality of teeth of the at least one ratchet racks when the compressible bellow portion is disposed in a non-stressed operative orientation.

Preferably, mounting of the injection port to the pumping assembly urges sliding of the first portion of the time duration start switch relative to the second portion thereof and thereby activates the time duration start switch. Further preferably, the injection port is independent of the pumping assembly. Still preferably, the elapsed time to initiation of injection is stored within a memory of the pumping assembly and upon connection of the pumping assembly to the different injection port, the timer proceeds counting the remaining time to initiation of injection. Yet further preferably, the time count continues when the pumping assembly is disconnected from the injection port.

In accordance with an embodiment of the present invention, the fluid pressure generator is prevented from discharging gas before the timer counts a pre-determined amount of time since activation of the time duration start switch.

Preferably, upon activation of the fluid pressure generator, gas is discharged, thereby building-up pressure between the first piston assembly and the second piston assembly, thus urging disengagement of the tooth from one of the plurality of teeth of the at least one ratchet racks and allowing axial displacement of the first piston assembly in a medicament pumping direction. Further preferably, actuation of the fluid pressure generator produces a gas volume between the first piston assembly and the second piston assembly, that is operative for ejecting the medicament from the reservoir assembly. Still further preferably, when the pumping assembly is disconnected from the injection port during injection of the medicament through the medicament injection pathway, the injection process is stopped and automatically resumes only upon connection of the pumping assembly to the different injection port.

Preferably, the pumping assembly is adapted for pumping a volume of approximately 1 ml of the medicament through the injection port within a time duration of less than approximately 5 minutes.

In accordance with an embodiment of the present invention, a pump assembly useful for pumping a medicament through an injection port which is removably mountable onto a patient body surface, the pump assembly including: a piston pumping assembly adapted for pumping the medicament through the injection port, the piston pumping assembly including: a fluid pressure generator; at least one piston which is driven in a medicament pumping displacement direction by fluid pressure generated by the fluid pressure generator; and a piston displacement controller preventing displacement of the piston for pumping the medicament through the injection port until the fluid pressure reaches a predetermined pressure level.

Preferably, the pumping assembly is removably mountable onto the injection port both prior to and following pumping of at least a portion of the medicament through the injection port. Further preferably, the piston pumping assembly is removable from the injection port and attachable to a different injection port without resulting in undesired pumping of the medicament through the injection port. Still further preferably, the piston pumping assembly is adapted for pumping the medicament through the injection port at a given time. Yet further preferably, the given time is measured from mounting of the pumping assembly onto the injection port.

In accordance with an embodiment of the present invention, the piston pumping assembly includes a timer which is actuated by a time duration start switch, the time duration start switch being actuated by mounting of the piston pumping assembly onto the injection port. Preferably, the piston pumping assembly includes a top housing portion, and a bottom housing portion fixedly mounted to the top housing portion and defining an interior volume therebetween, which encloses a reservoir assembly extending along a longitudinal axis and a circuit and switch assembly, an underside of said bottom housing portion is at least partially covered by an adhesive layer, which is adapted to be secured to said patient body surface.

Further preferably, the reservoir assembly includes a barrel assembly having an interior volume, a forward closed end and a rearward open end, the barrel assembly also includes a needle hub extending forwardly from the closed end, and a needle, which is partially inserted into the needle hub.

In accordance with an embodiment of the present invention, the piston displacement controller includes at least a portion of the piston, which is operatively associated with a ratchet assembly when the piston displacement controller is disposed in an actuated operative orientation. Alternatively, the piston displacement controller includes an electrical valve incorporated with a needle, which is activated upon receiving of an electrical signal indicating actuation of the fluid pressure generator. Further alternatively, the piston displacement controller includes a mechanical valve operatively coupled with a needle, which is activated by pressure exerted thereon following gas discharge from the fluid pressure generator.

Preferably, the barrel assembly having at least one protrusion configured to receive the ratchet assembly therewithin, which communicates with the interior volume of the barrel assembly. Further preferably, the reservoir assembly encloses the piston, a second piston and the fluid pressure generator enclosed therebetween at least in some of the operative orientations of the pump assembly. Still preferably, the ratchet assembly includes a compressible bellow portion and a ratchet tooth fixed thereto and having a straight edge and a tapered edge, the bellow portion is operative to be compressed upon exertion of force on the ratchet assembly.

Yet further preferably, the piston has a forwardly facing surface and a rearwardly facing surface and the second piston assembly has a forwardly facing surface and a rearwardly facing surface.

In accordance with an embodiment of the present invention, the first piston includes at least one ratchet rack, extending longitudinally from a location adjacent to the forwardly facing surface to a location rearwardly disposed with respect to rearwardly facing surface, the at least one ratchet rack includes a plurality of ratchet teeth, each defining a straight edge and a tapered edge.

Preferably, the fluid pressure generator has at least one opening for facilitating discharge of gas therefrom following activation of the fluid pressure generator. Further preferably, the piston displacement controller is operative both for restraining forward displacement of the piston in certain operative orientations and for fluid tight sealing of gas that is generated by the fluid pressure generator in other operative orientations. Still preferably, forward displacement of the piston is prevented by means of engagement of the tooth of said ratchet assembly with one of the plurality of teeth of the at least one ratchet racks when the compressible bellow portion is disposed in a non-stressed operative orientation. Yet further preferably, upon activation of the fluid pressure generator, gas is discharged, thereby building-up pressure between the piston and the second piston, thus urging disengagement of the tooth from one of the plurality of teeth of the at least one ratchet racks and allowing axial displacement of the piston in said medicament pumping direction.

In accordance with an embodiment of the present invention, a pump assembly useful for pumping a medicament through an injection port which is removably mountable onto a patient body surface, the pump assembly including a double piston pumping assembly adapted for pumping the medicament through the injection port, the piston pumping assembly having first and second pistons.

Preferably, the first and second pistons both move in a same direction during supply of the medicament to the pumping assembly and the first and second pistons move in mutually operative orientations during pumping of the medicament through the injection port. Alternatively, the first and second pistons both move in a same direction during supply of the medicament to the pumping assembly, whereas during pumping of the medicament through the injection port, one of the pistons remains static and the other one of the pistons moves in a medicament pumping direction.

In accordance with an embodiment of the present invention, the double piston pumping assembly is removably mountable onto the injection port both prior to and following pumping of at least a portion of the medicament through the injection port.

Preferably, the double piston pumping assembly is removable from the injection port and attachable to a different injection port without resulting in undesired pumping of the medicament through the injection port. Further preferably, the double piston pumping assembly includes a timer which is actuated by a time duration start switch, the time duration start switch being actuated by mounting of the double piston pumping assembly onto the injection port. Still further preferably, the double piston pumping assembly is removably mountable onto the injection port both prior to and following pumping of at least a portion of the medicament through the injection port. Yet further preferably, the double piston pumping assembly is removable from the injection port and attachable to a different injection port without resulting in undesired pumping of the medicament through the injection port.

In accordance with an embodiment of the present invention, the double piston pumping assembly includes a timer which is actuated by a time duration start switch, the time duration start switch being actuated by mounting of the double piston pumping assembly onto the injection port.

Preferably, the double piston pumping assembly includes a top housing portion, and a bottom housing portion fixedly mounted to the top housing portion and defining an interior volume therebetween, which encloses a reservoir assembly extending along a longitudinal axis and a. circuit and switch assembly, an underside of said bottom housing portion is at least partially covered by an adhesive layer, which is adapted to be secured to said patient body surface.

Further preferably, the reservoir assembly encloses the first piston, the second piston and a fluid pressure generator enclosed therebetween at least in some of the operative orientations of the double piston pump assembly. Still further preferably, the first piston has a forwardly facing surface and a rearwardly facing surface and the second piston has a forwardly facing surface and a rearwardly facing surface. Yet further preferably, the forwardly facing surface of the first piston abuts the closed end of the reservoir assembly when the pump assembly is empty of the medicament.

In accordance with an embodiment of the present invention, the interior volume of the reservoir assembly is entirely empty of fluid before filling thereof, thereby obviating the need for priming of the reservoir assembly after filling the reservoir assembly with the medicament.

Preferably, the rearwardly facing surface of the first piston abuts the forwardly facing surface of the second piston when the fluid pressure generator is disposed in a non-activated operative orientation. Further preferably, actuation of the fluid pressure generator produces a gas volume between the first pistol d the second piston, that is operative for ejecting the medicament from the reservoir assembly.

In accordance with an embodiment of the present invention, a pump assembly useful for pumping a medicament through an injection port which is removably mountable onto a patient body surface, the pump assembly including: a piston pumping assembly adapted for pumping the medicament through the injection port, the piston pumping assembly including: a fluid pressure generator; at least one piston which is driven in a medicament pumping displacement direction by fluid pressure generated by the fluid pressure generator; and a piston location sensor for sensing a position of at least one of the at least one piston, thereby indicating completion of injection.

Preferably, the completion of injection is visually indicated. Alternatively, or additionally, the completion of injection is audibly indicated. Alternatively, or additionally, the completion of injection is indicated in a tactile manner.

In accordance with an embodiment of the present invention, the piston location sensor is a proximity sensor. Alternatively, the piston location sensor is an optical sensor. Further alternatively, the piston location sensor is a magnetic sensor. Still further alternatively, the piston location sensor is a switch.

In accordance with an embodiment of the present invention, a pump assembly useful for pumping a medicament through an injection port which is removably mountable onto a patient body surface, the pump assembly including: a pumping assembly adapted for pumping the medicament through the injection port, a communicator enabling wireless data communication between the pumping assembly and a remote communications device.

Preferably, the wireless data includes at least one of the following: indication of injection initiation; indication of injection completion; dosage injected; time of injection; errors or occlusions detected during injection. Further preferably, the remote communications device is operative for controlling the pumping assembly.

In accordance with an embodiment of the present invention, a pump assembly useful for pumping a medicament through an injection port which is removably mountable onto a patient body surface, the pump assembly including: a pumping assembly adapted for pumping the medicament through the injection port at an injection time; an environmental sensor for sensing environmental parameters relevant to timing of injection by the pumping assembly; and a controller receiving an input from the environmental sensor and providing an operational control input to the pumping assembly.

Preferably, the environmental sensor is operative for identifying at least one of the following situations: patient is within a high-level radiation area; patient is at a high altitude with low level of oxygen; patient is exposed to a hazardous chemical environment. Further preferably, upon identifying any of these situations, automatic initiation of injection is enabled either before or after the scheduled injection time.

In accordance with an embodiment of the present invention, a method of supplying a medicament to a patient including the steps of: removably mounting an injection port onto a body surface of a patient; thereafter removably mounting a pumping assembly onto the injection port; and thereafter operating the pumping assembly to pump the medicament through the injection port.

Preferably, the pumping assembly is removable from the injection port both prior to and following pumping of at least a portion of the medicament through the injection port. Further preferably, the pumping assembly is removable from the injection port and attachable to a different injection port without resulting in undesired pumping of the medicament through the injection port.

In accordance with an embodiment of the present invention, a medicament supply kit including: an injection port which is removably mountable onto a body surface of a patient; and a timed pumping assembly which is separate from and removably engageable with the injection port. Preferably, the medicament supply kit also includes a filling port assembly removably mountable to the timed pumping assembly in order to supply the medicament to the pumping assembly. Further preferably, the pumping assembly is removable from the injection port both prior to and following pumping of at least a portion of the medicament through the injection port.

Still further preferably, the pumping assembly includes a timer which is actuated by a time duration start switch, the time duration start switch being actuated by mounting of the pumping assembly onto the injection port.

### BRIEF DESCRIPTION OF THE DRAWINGS

The present invention will be understood and appreciated more fully from the following detailed description, taken in conjunction with the drawings in which:

FIG. 1 is a simplified pictorial illustration of a transfusion pump with a filling port assembly mounted thereto, constructed and operative in accordance with a preferred embodiment of the present invention;

FIGS. 2A & 2B are respective simplified downward-facing and upward-facing exploded view pictorial illustrations of the transfusion pump of FIG. 1;

FIGS. 3A, 3B and 3C are respective simplified exploded view sectional illustrations of the transfusion pump of FIG. 1 taken along respective lines A-A, B-B and C-C in FIG. 2A;

FIGS. 4A & 4B are respective simplified downward-facing and upward-facing pictorial illustrations of a top housing portion of the transfusion pump of FIG. 1;

FIGS. 5A, 5B and 5C are respective simplified sectional illustrations of the top housing portion of FIGS. 4A & 4B, taken along respective lines A-A, B-B and C-C in 4A;

FIGS. 6 & 7 are respective simplified upward-facing pictorial and sectional illustrations of a bottom housing portion of the transfusion pump of FIG. 1, FIG. 7 being taken along lines A-A in FIG. 6;

FIGS. 8A-8D are respective simplified first and second pictorial side view illustrations and two sectional illustrations of circuit and switch assembly, forming part of the transfusion pump of FIG. 1, FIGS. 8C and 8D are taken along respective lines A-A, B-B in FIG. 8A;

FIG. 9 is a simplified pictorial illustration of a reservoir assembly, forming part of the transfusion pump of FIG. 1;

FIG. 10 is a simplified exploded view illustration of the reservoir assembly of FIGS. 9;

FIGS. 11A, 11B and 11C are respective simplified sectional illustrations of the reservoir assembly of FIG. 9, taken along respective lines A-A, B-B and C-C in FIG. 10;

FIG. 12 is a simplified pictorial illustration of a barrel assembly, forming part of the reservoir assembly of FIGS. 9-12C;

FIGS. 13A, 13B and 13C are respective simplified sectional illustrations of the barrel assembly of FIG. 12, taken along respective lines A-A, B-B and C-C in FIG. 12;

FIGS. 14 and 15 are respective simplified pictorial and sectional illustrations of a ratchet assembly, forming part of the reservoir assembly of FIGS. 9-11C, FIG. 15 being taken along lines A-A in FIG. 14;

FIGS. 16A and 16B are respective simplified first and second side view pictorial illustrations of a first piston assembly, forming part of the reservoir assembly of FIGS. 9-11C;

FIGS. 17A and 17B are respective simplified first and second side view sectional illustrations of the first piston assembly of FIGS. 16A & 16B, taken along respective lines A-A and B-B in FIG. 16A;

FIGS. 18A and 18B are respective simplified first and second side view pictorial illustrations of a second piston assembly, forming part of the reservoir assembly of FIGS. 9-11C;

FIGS. 19A and 19B are respective simplified first and second side view sectional illustrations of the second piston assembly of FIGS. 18A & 18B, taken along respective lines A-A and B-B in FIG. 18A;

FIGS. 20A-20E are respective simplified forward-facing, rearward-facing and downwardly-facing pictorial illustrations of a partial sub-assembly of the reservoir assembly of

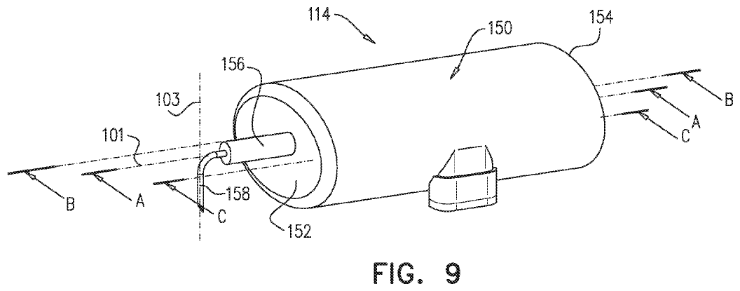

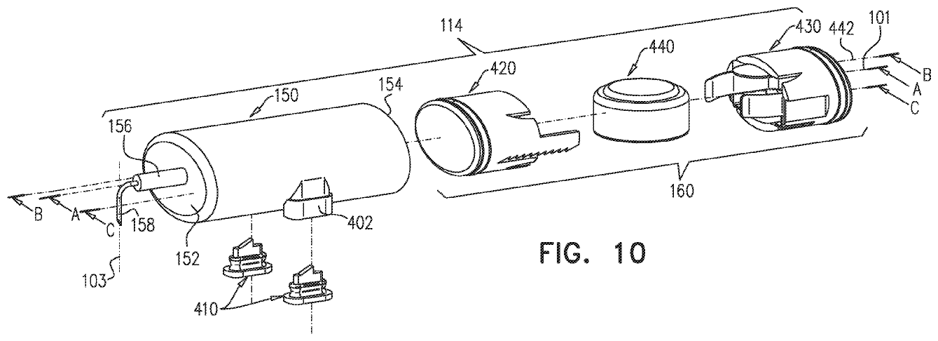

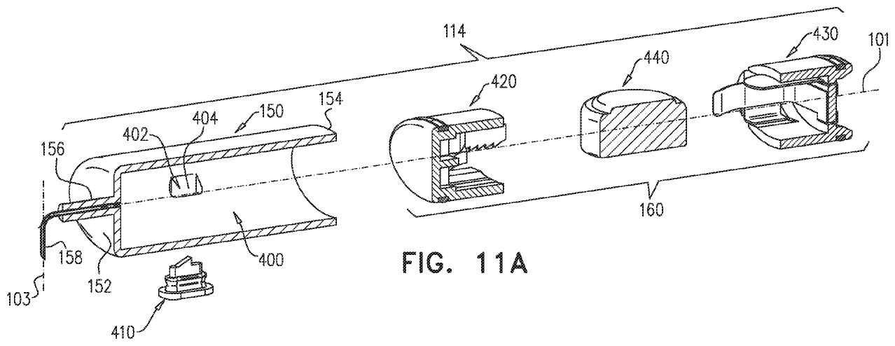

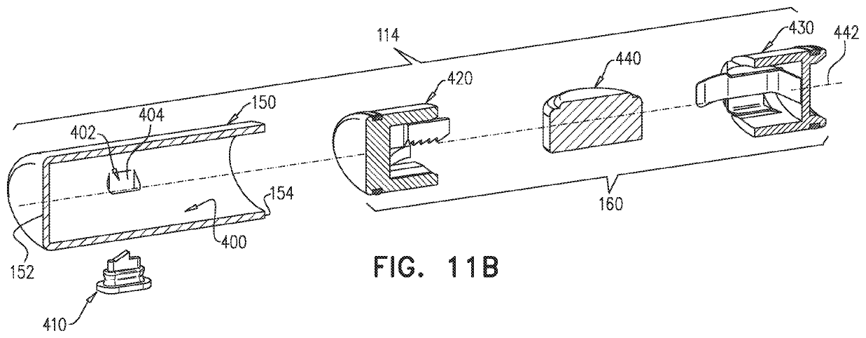

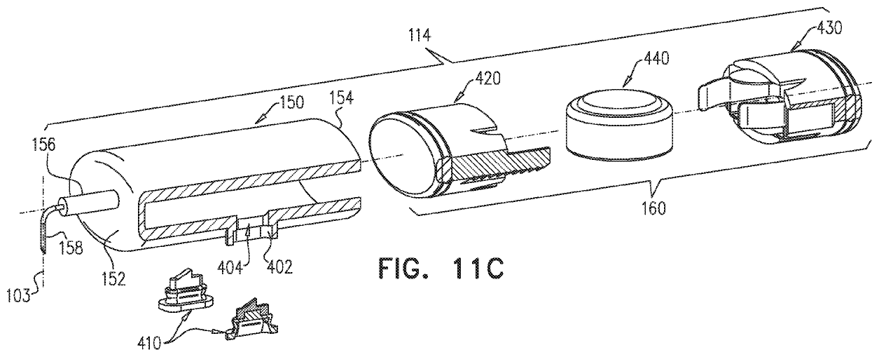

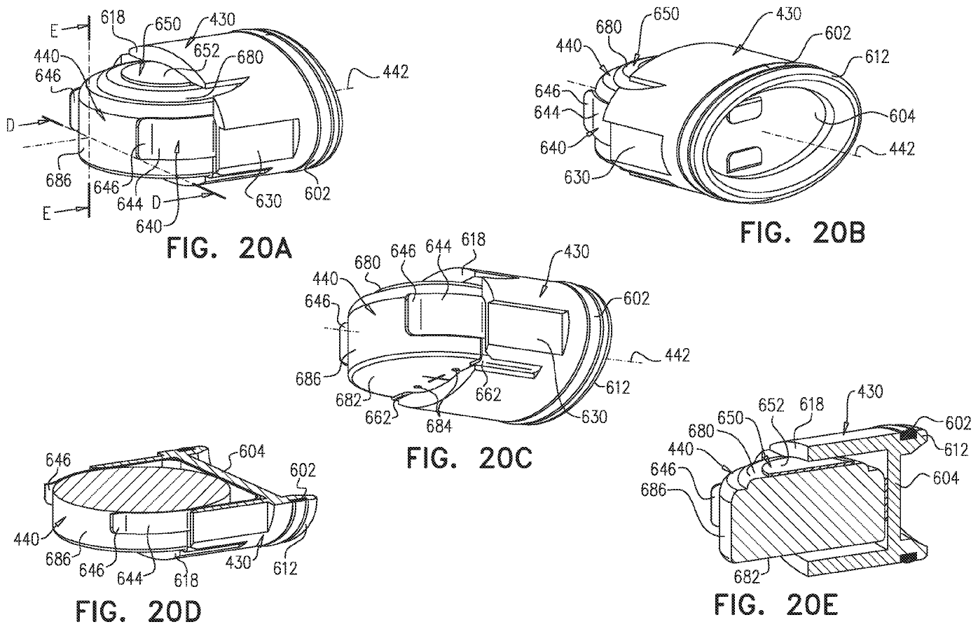

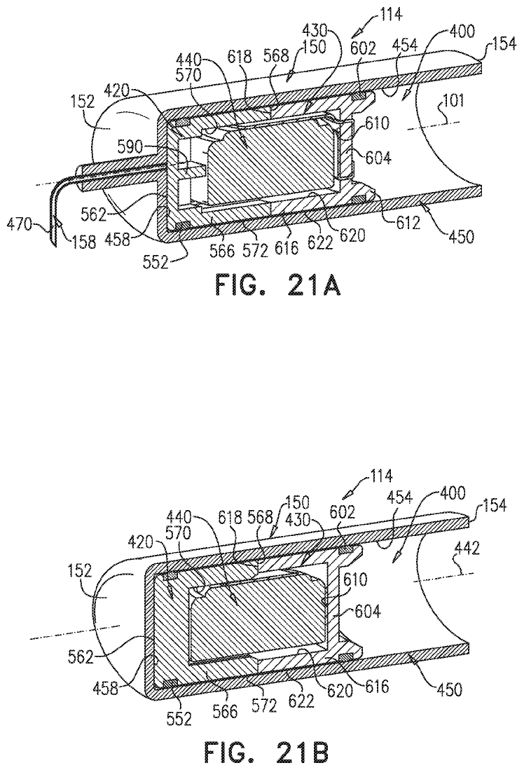

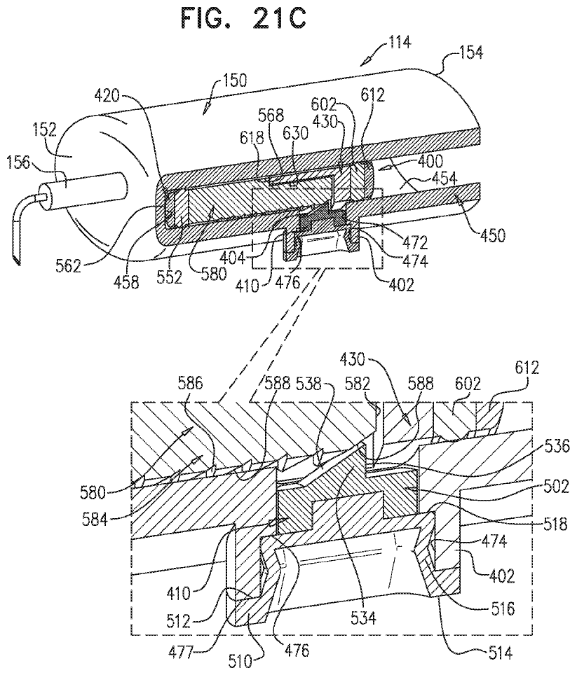

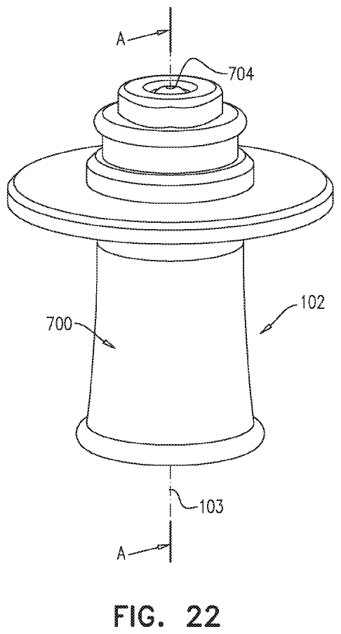

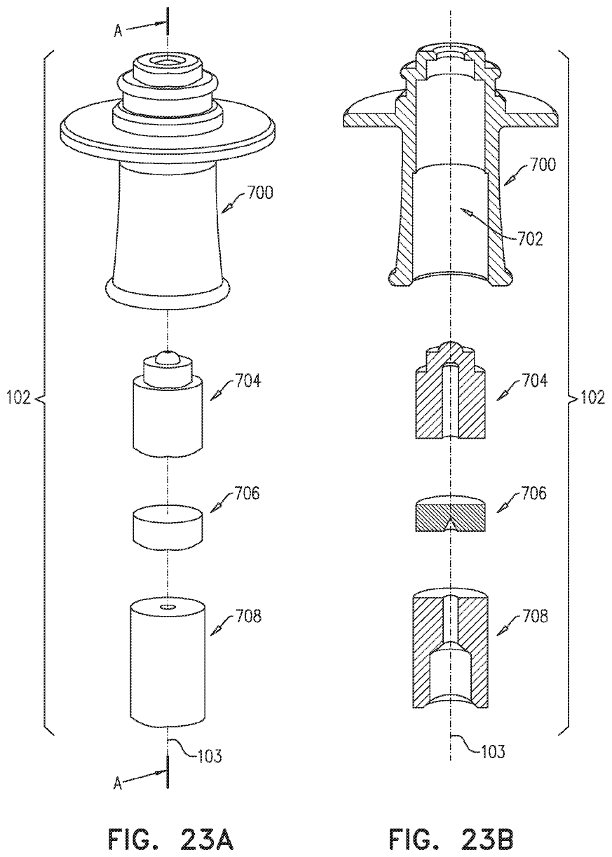

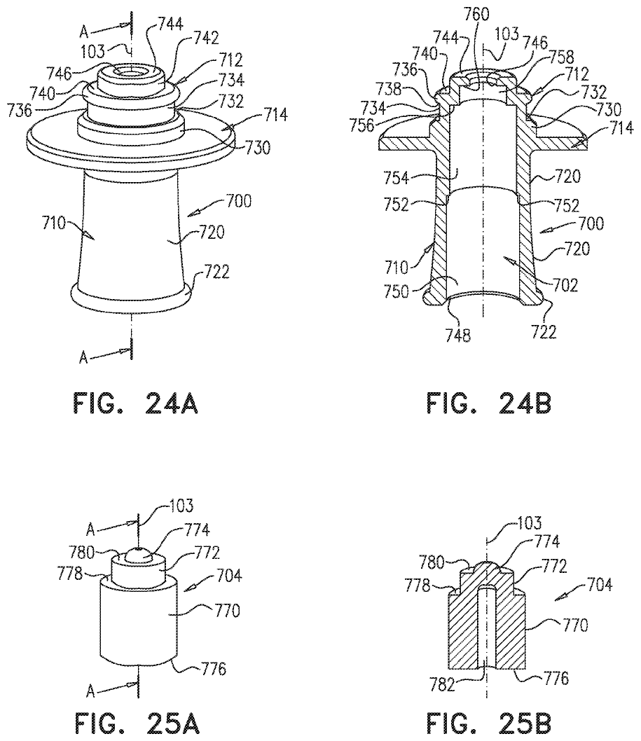

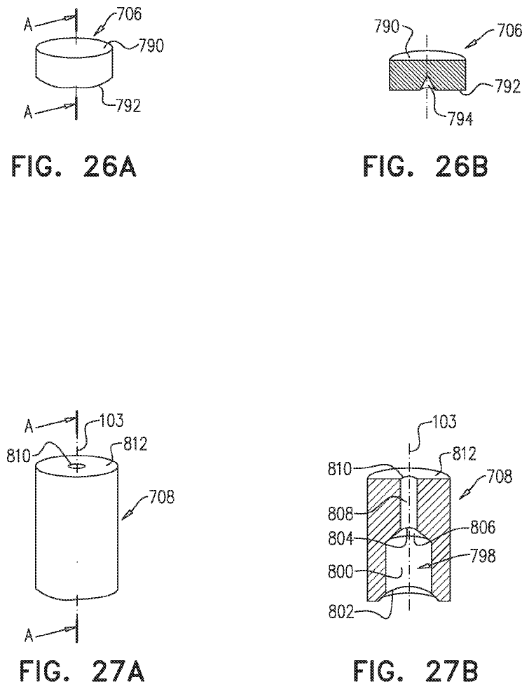

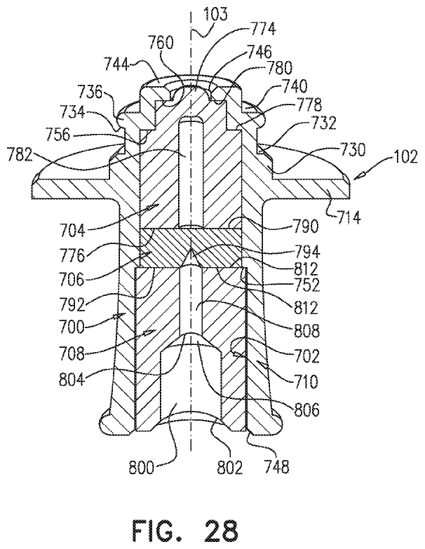

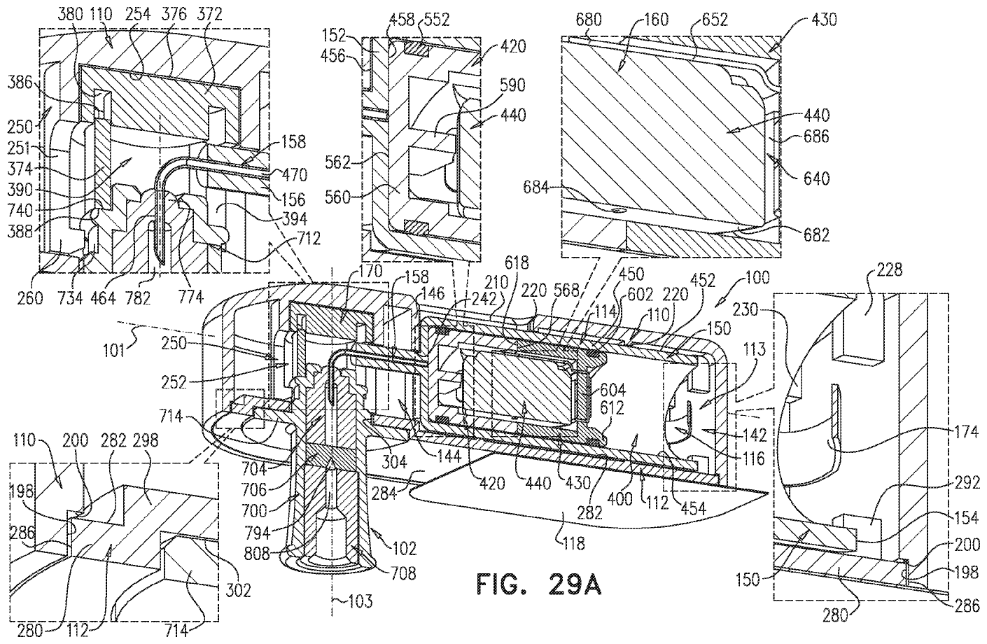

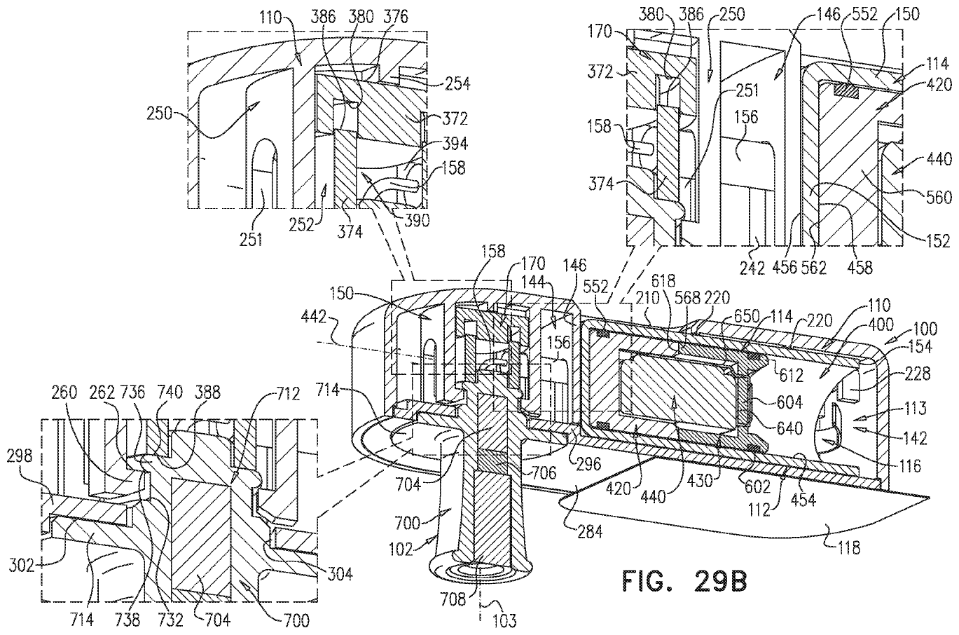

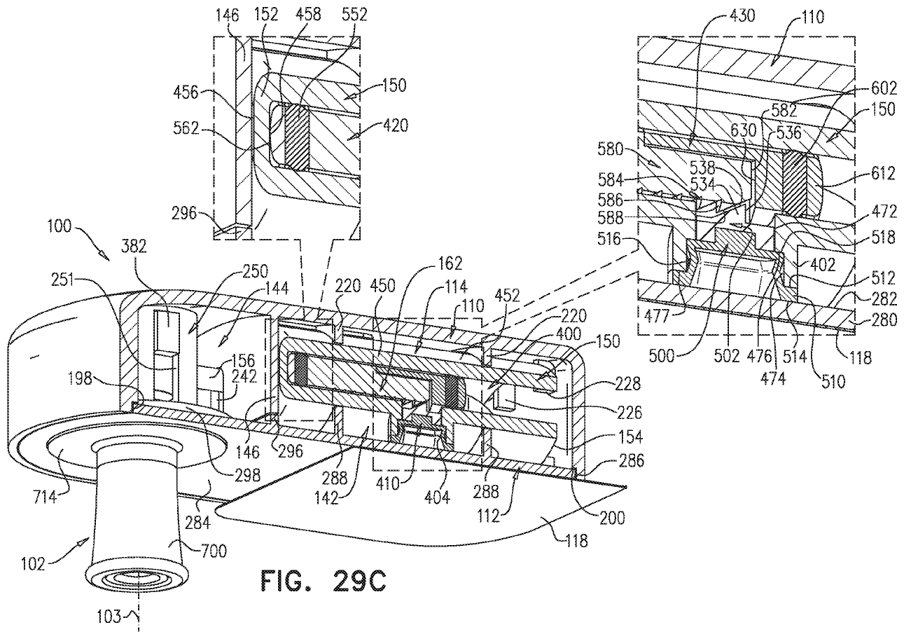

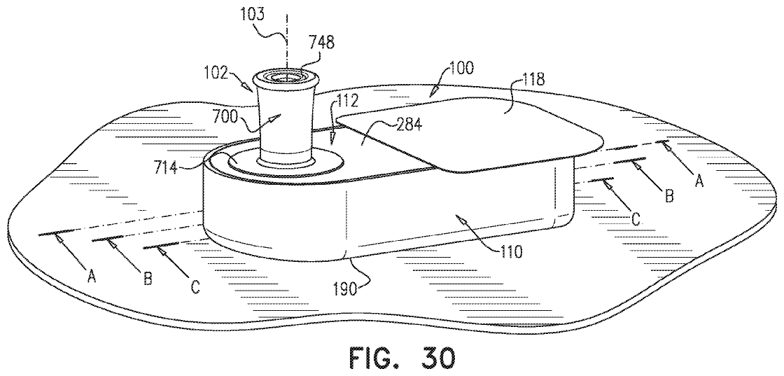

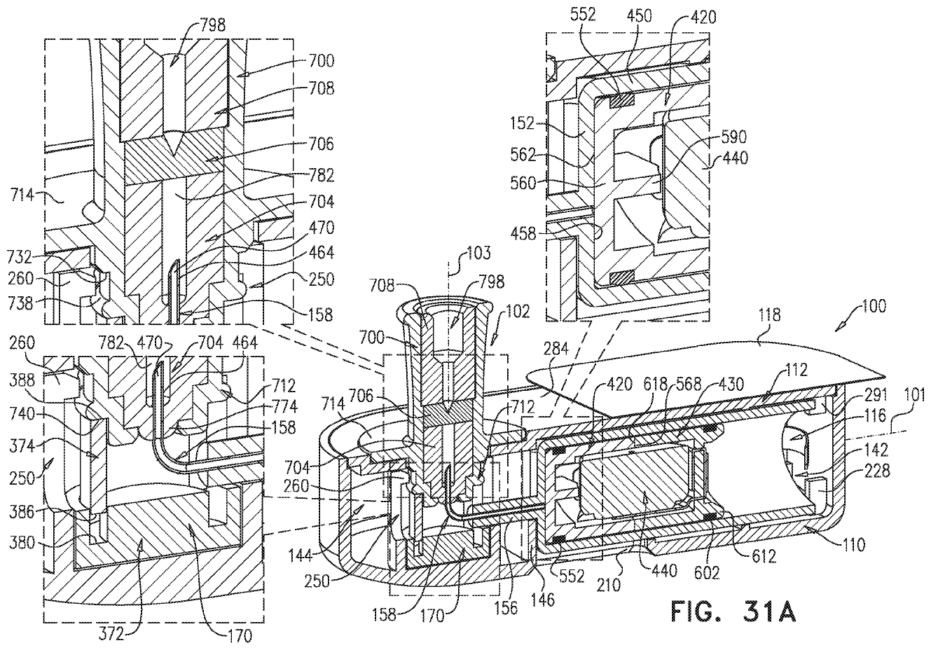

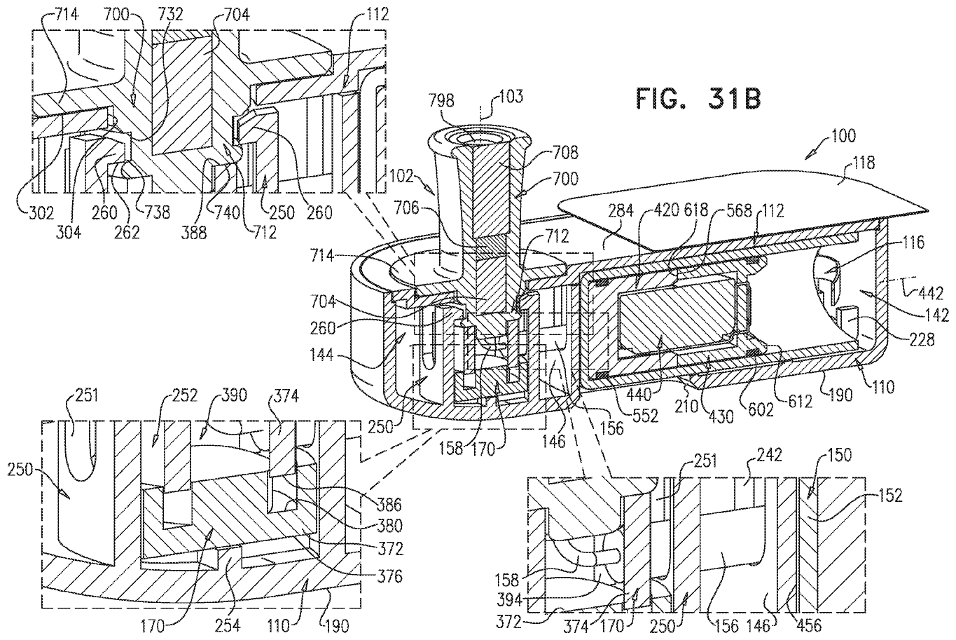

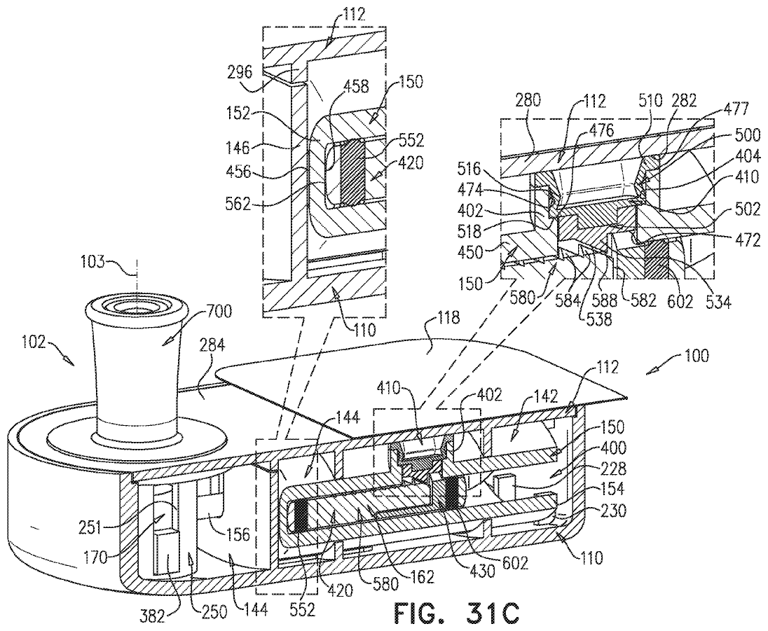

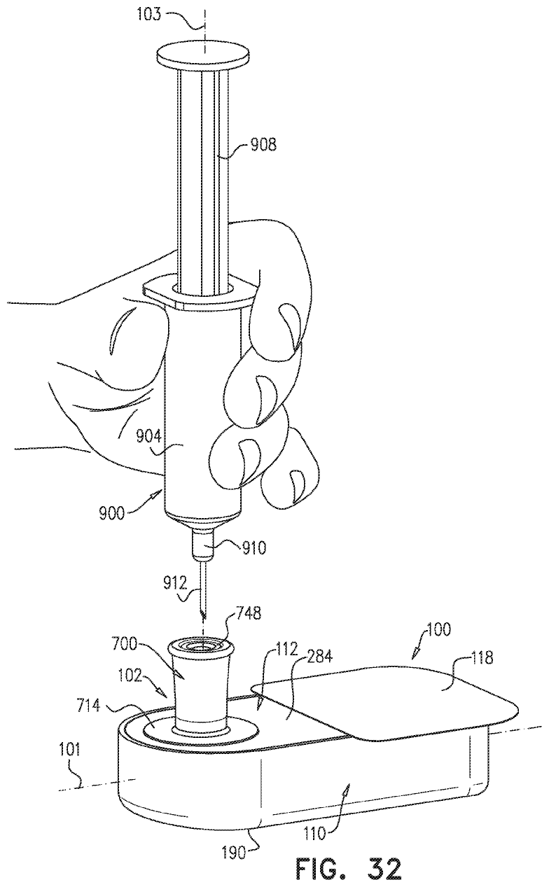

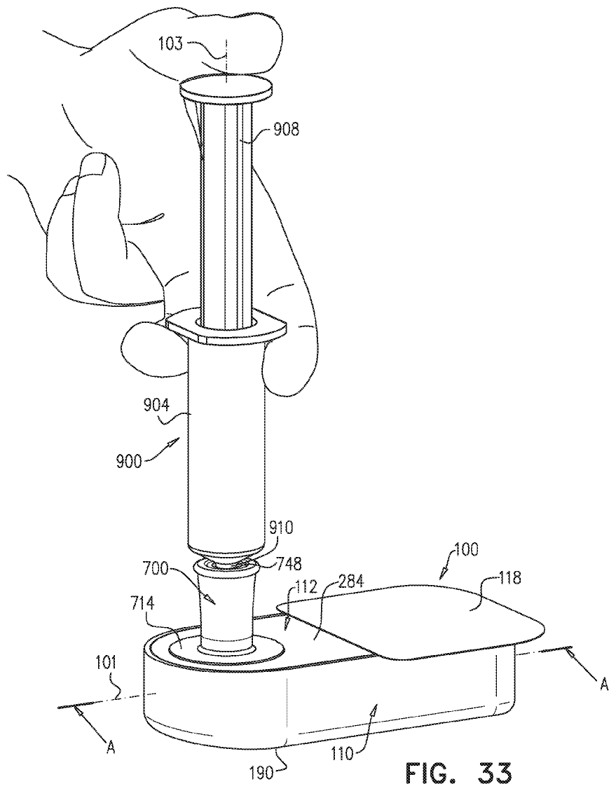

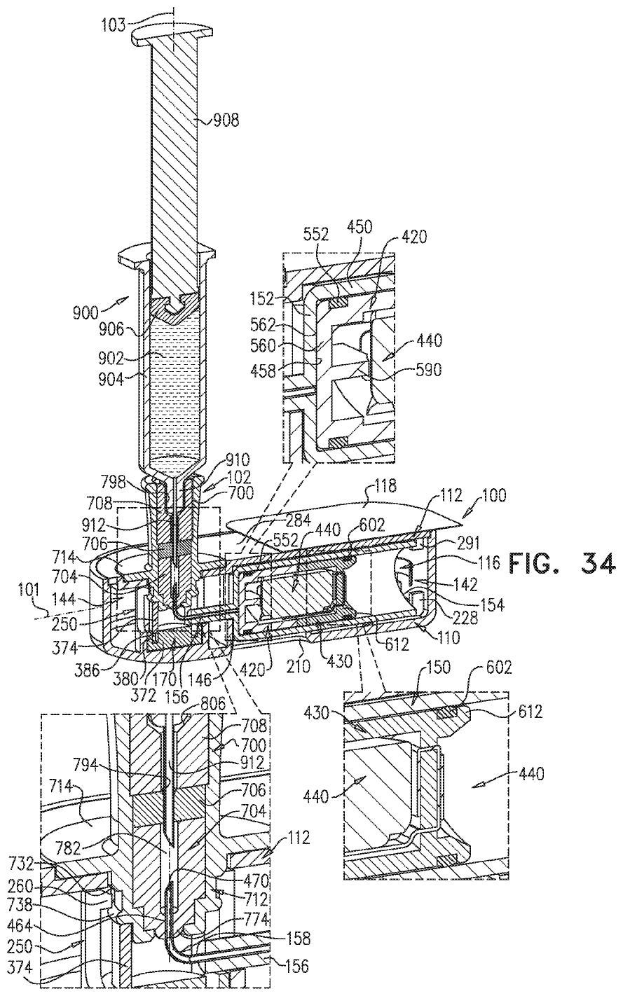

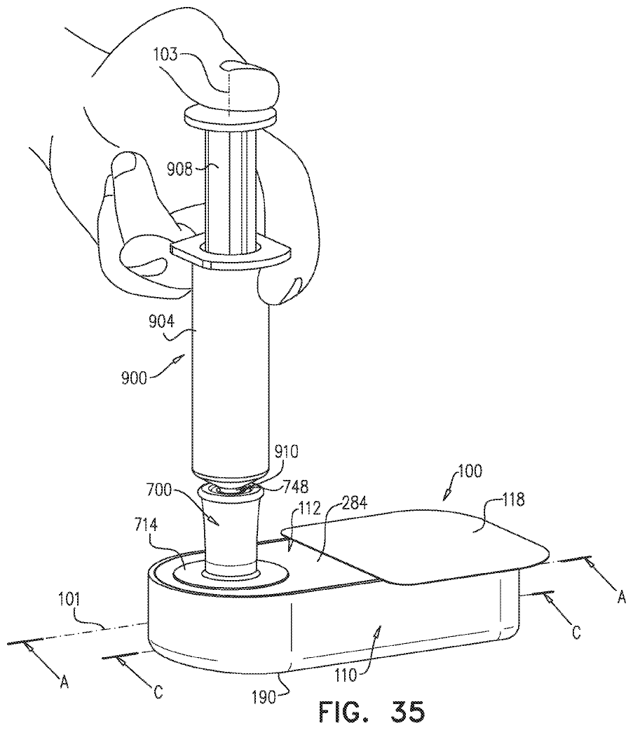

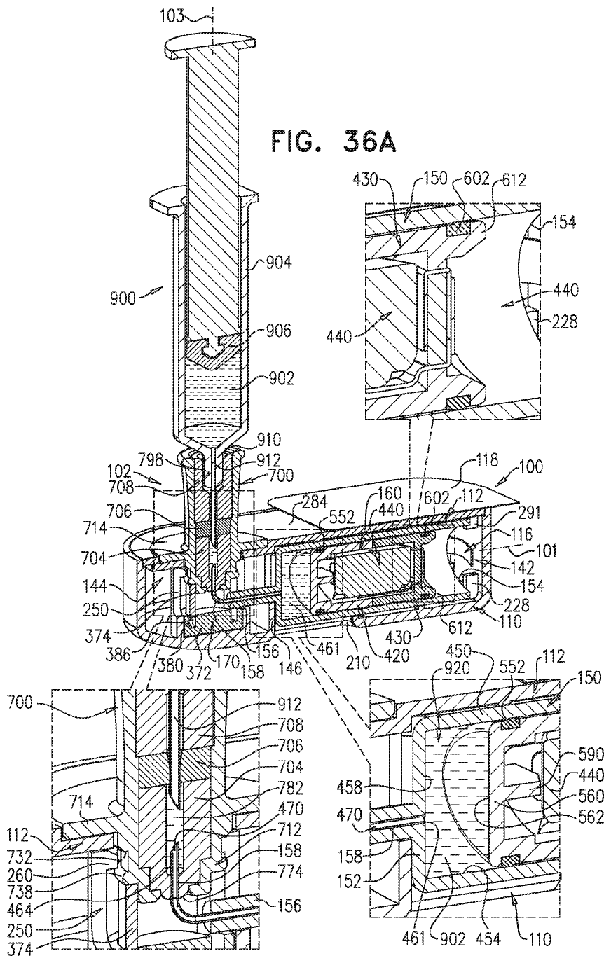

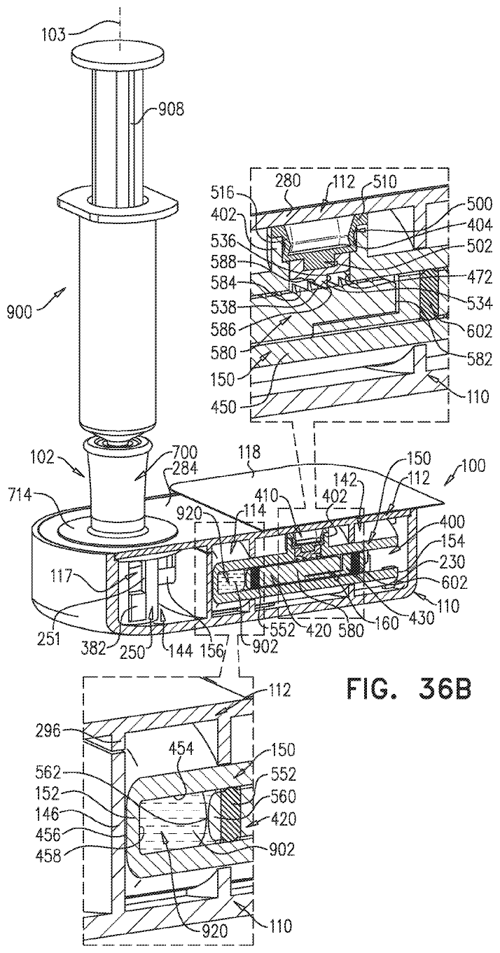

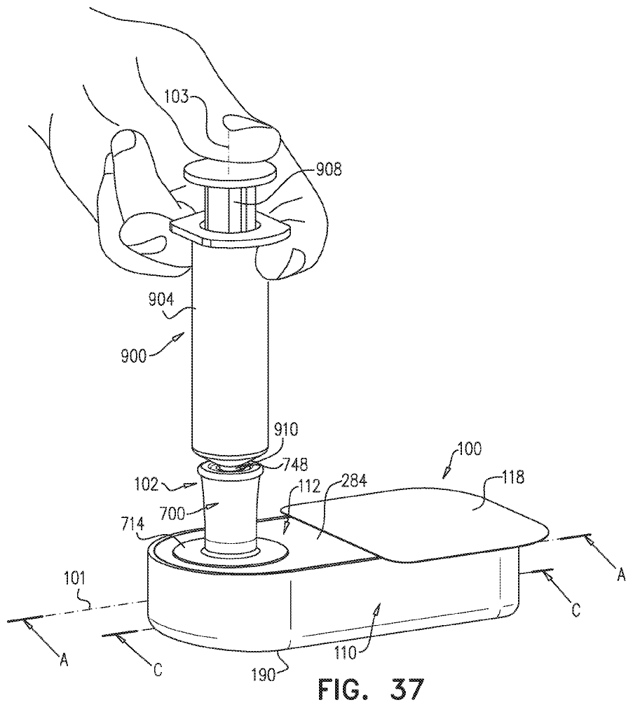

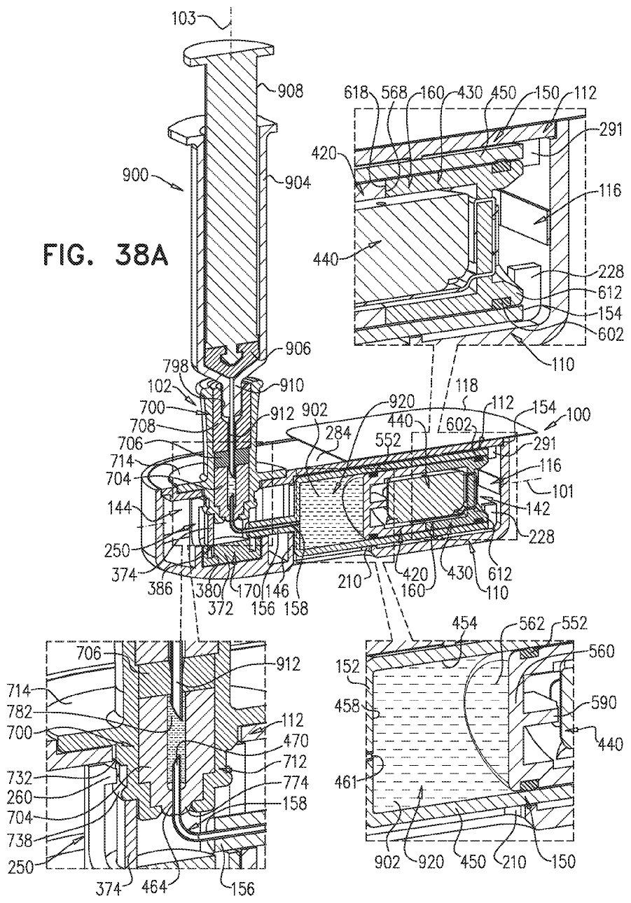

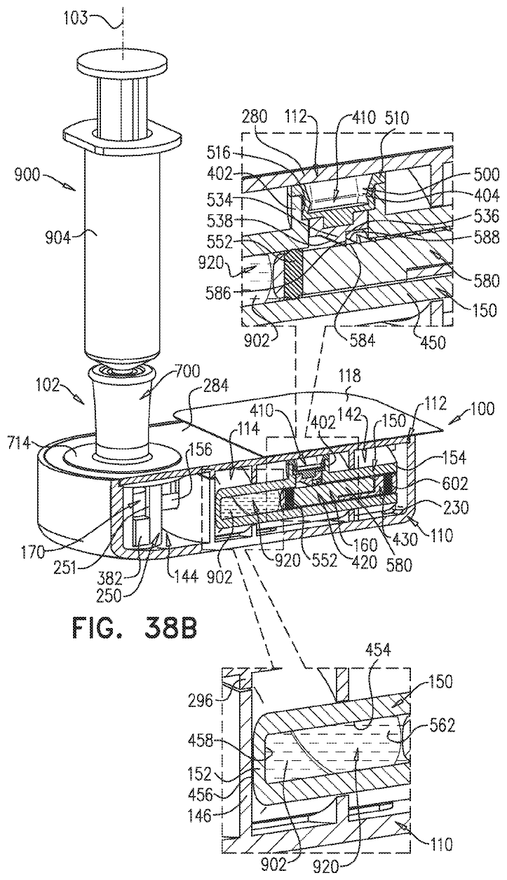

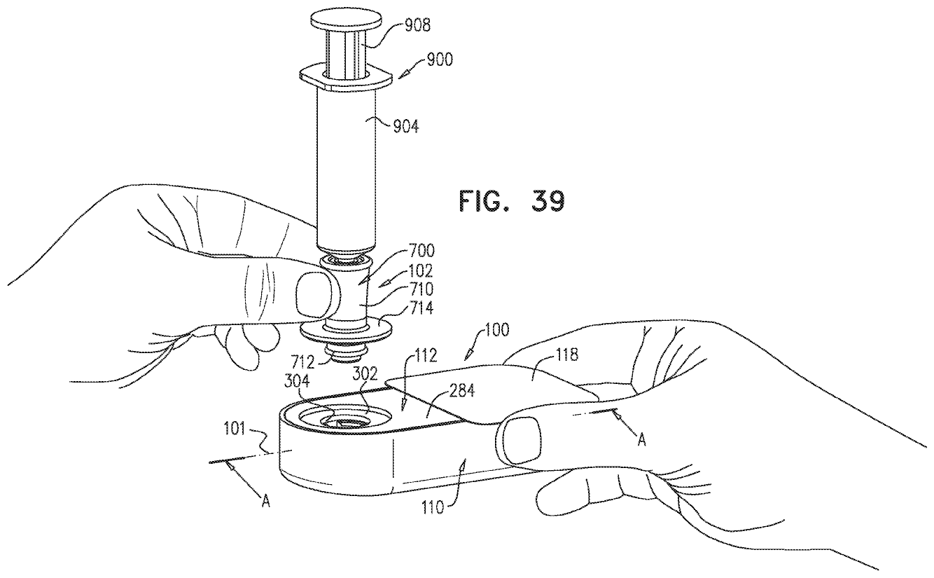

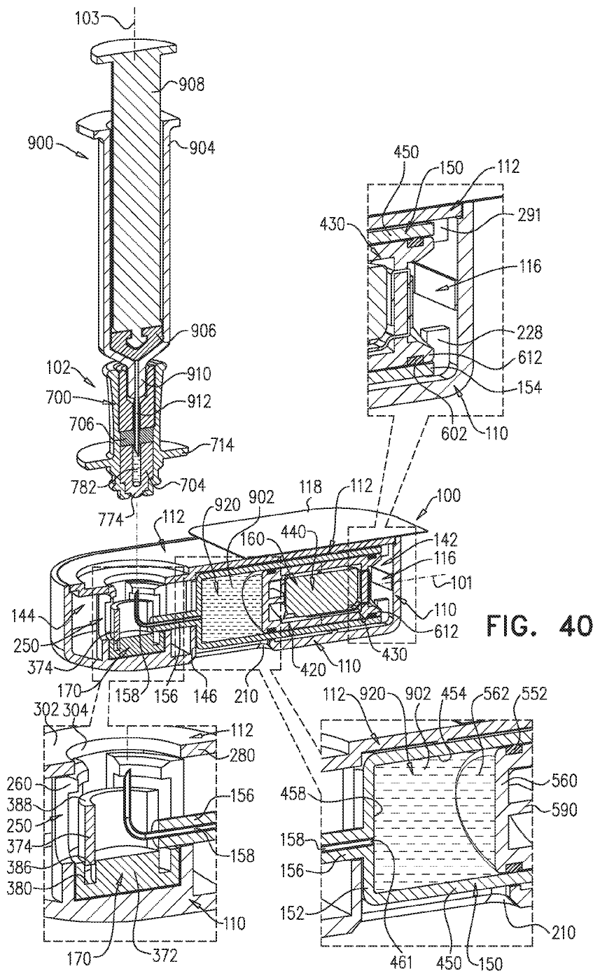

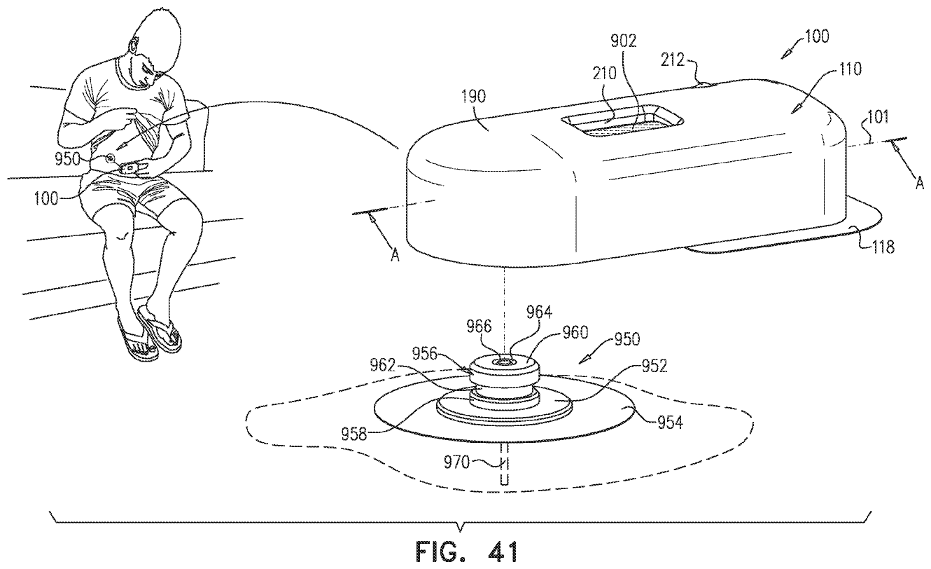

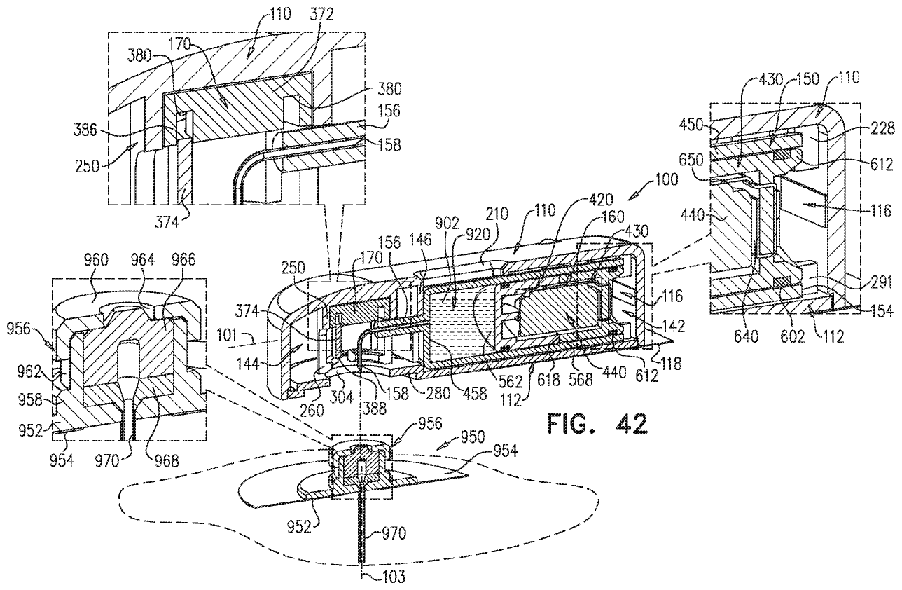

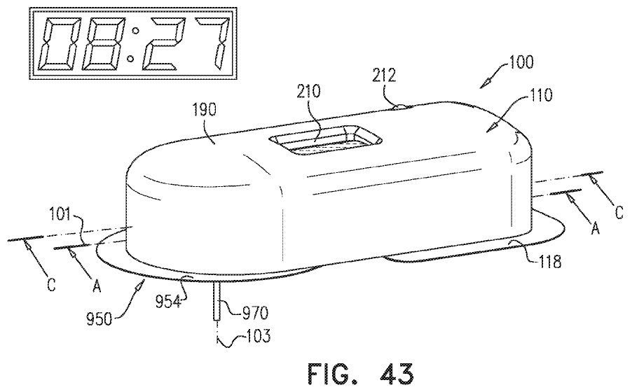

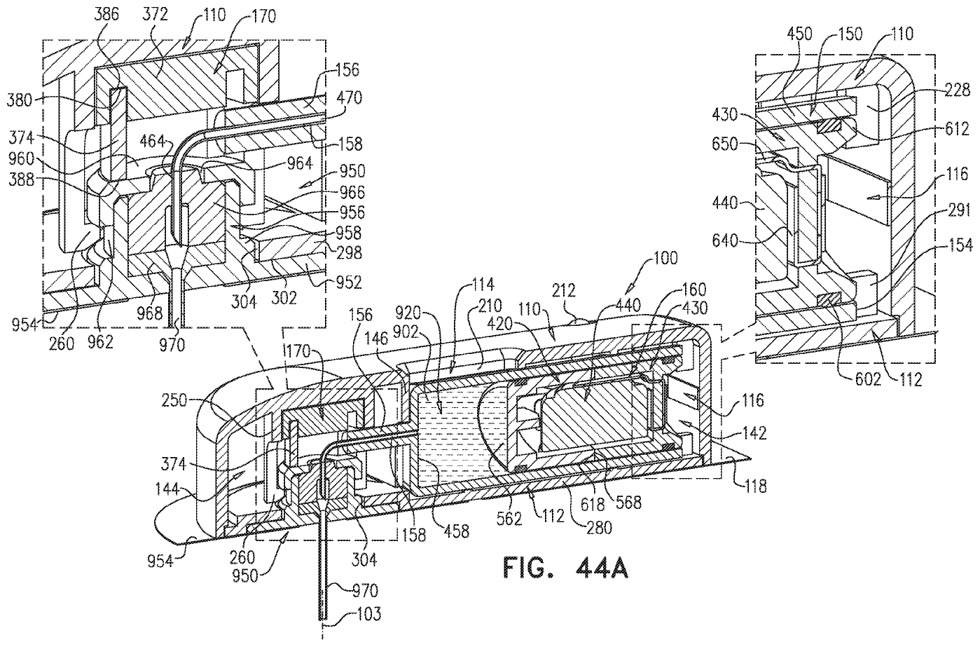

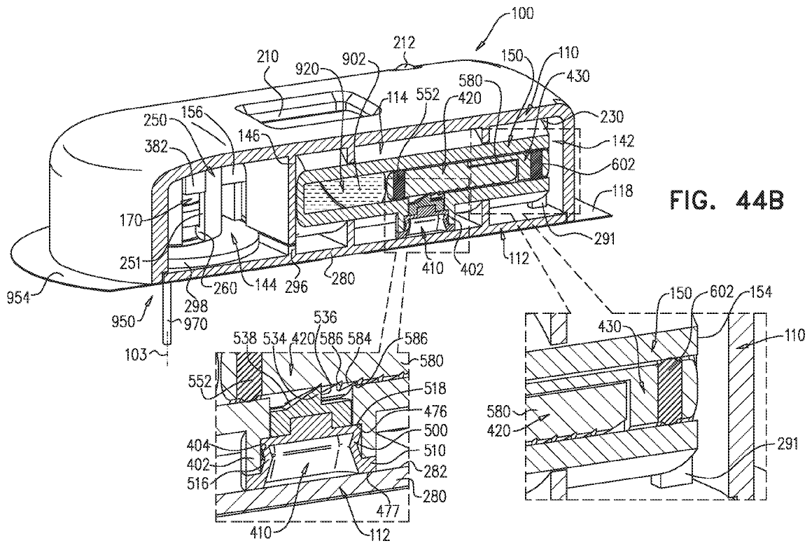

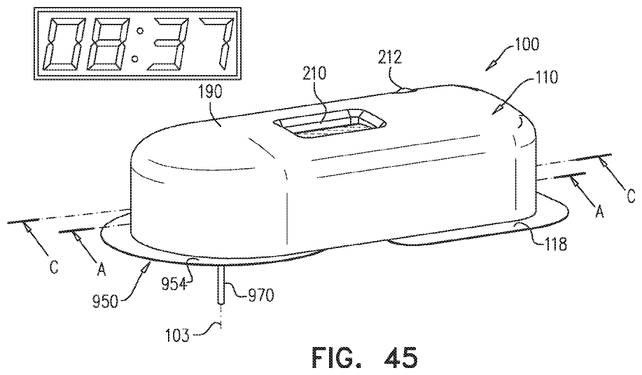

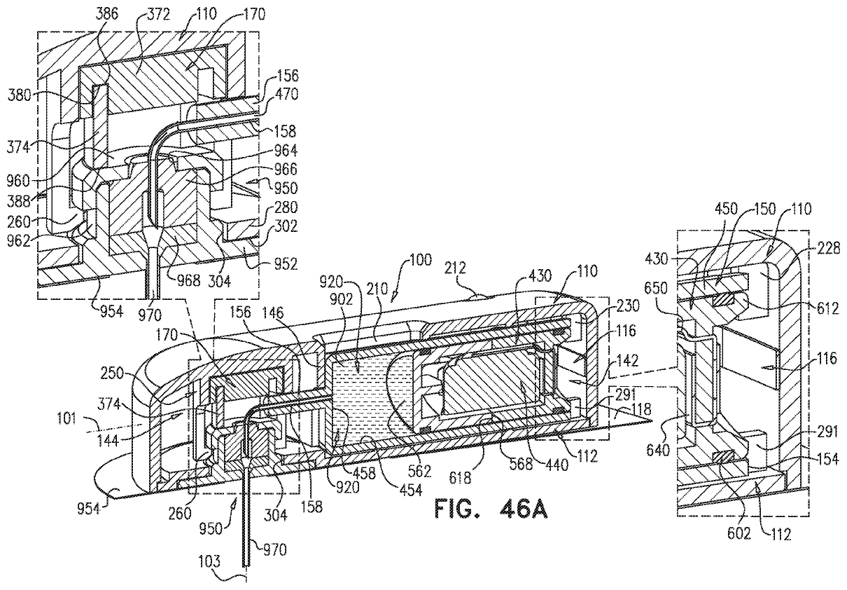

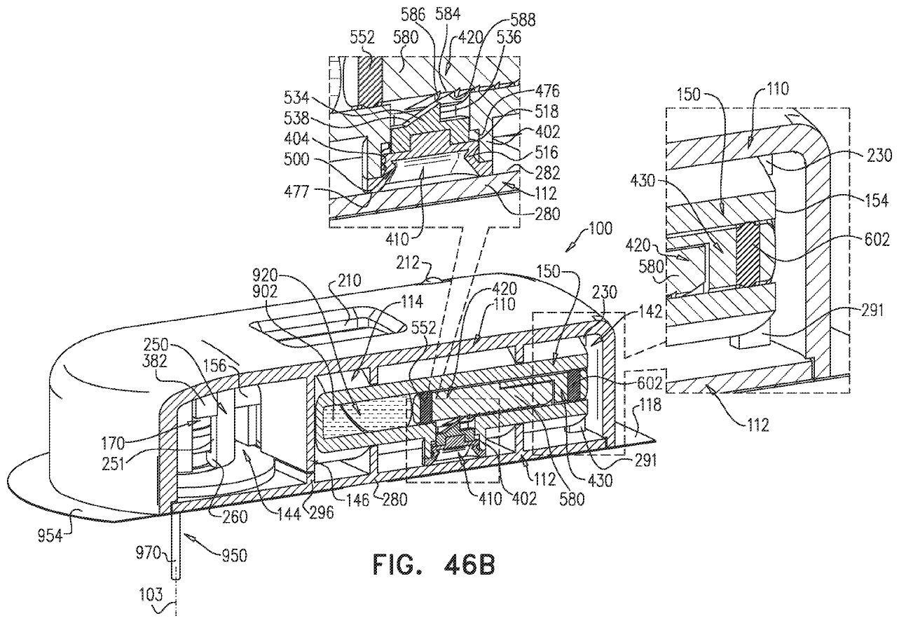

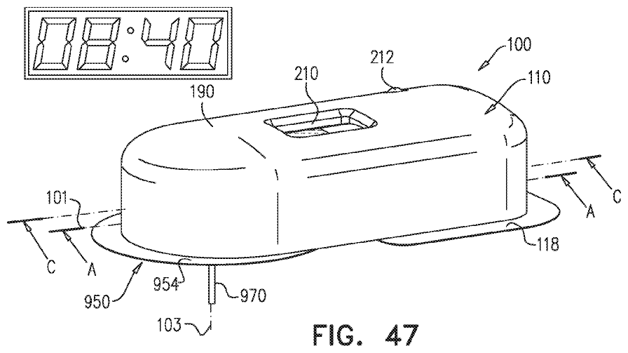

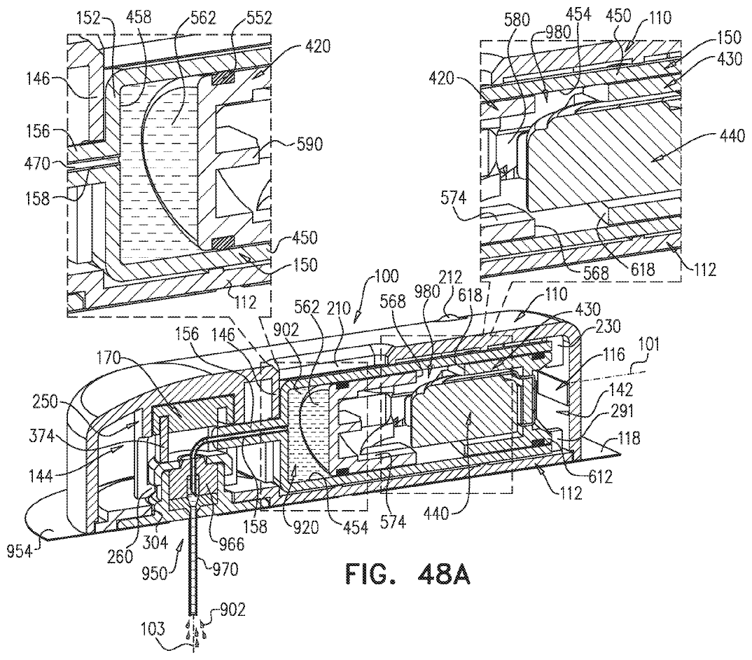

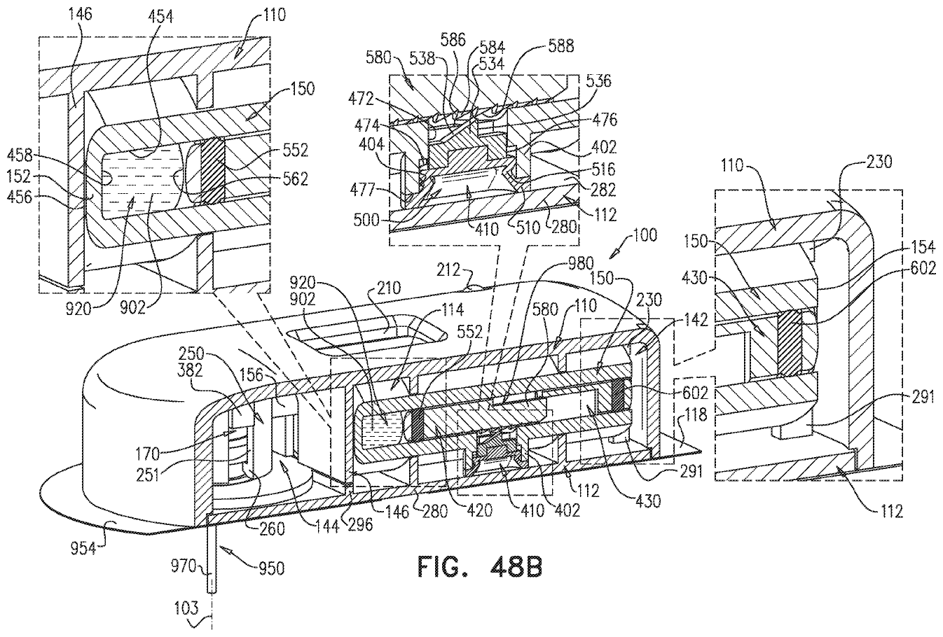

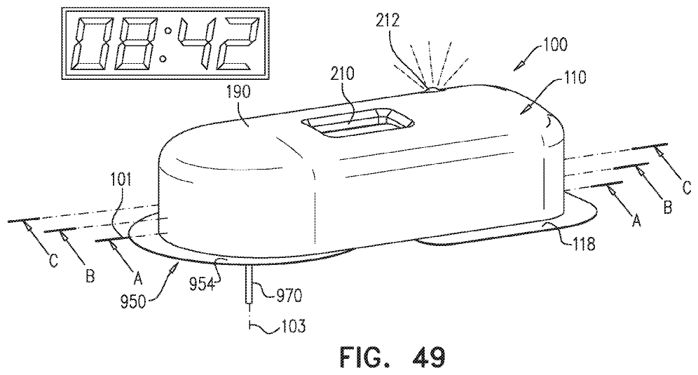

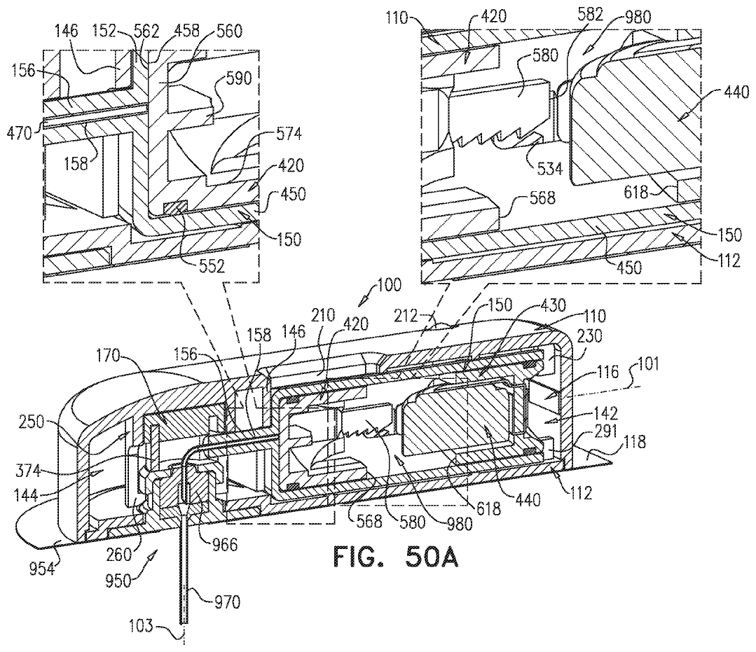

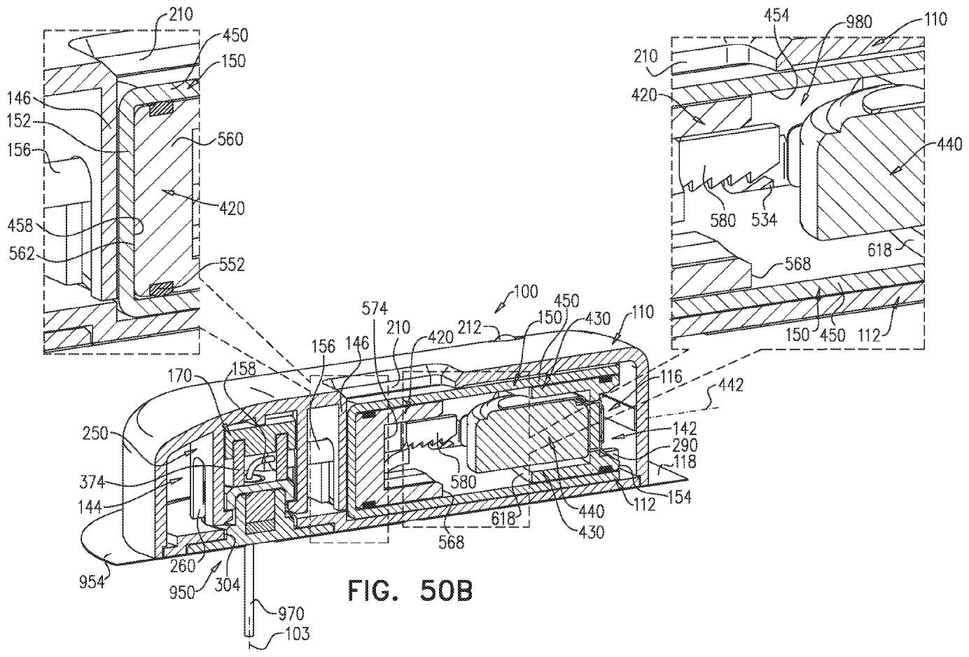

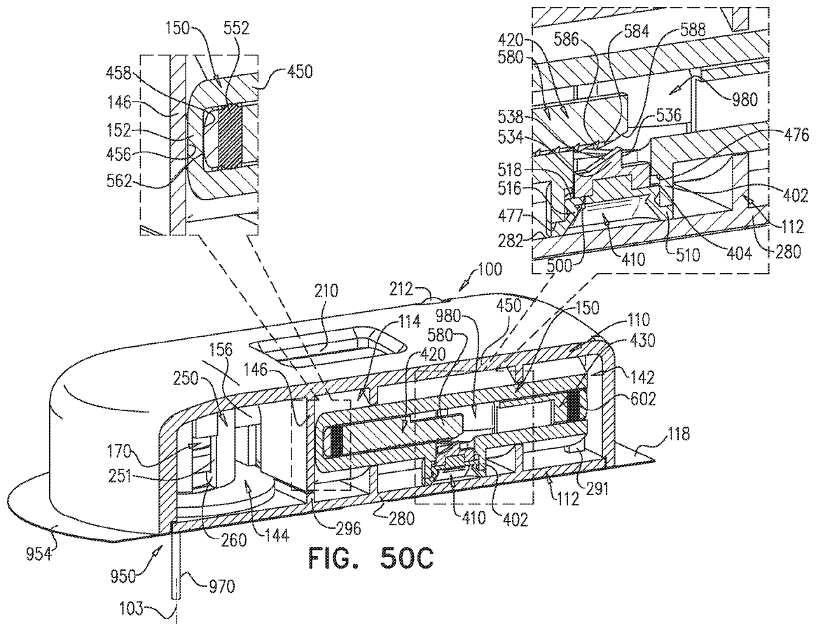

FIG. 9 and two sectional illustrations of the partial sub-assembly of the reservoir assembly, FIGS. 20D and 20E are taken along respective lines D-D and E-E in FIG. 20A;

FIGS. 21A, 21B and 21C are respective simplified sectional illustrations of the reservoir assembly of FIG. 9, taken along respective lines A-A, B-B and C-C in FIG. 9;

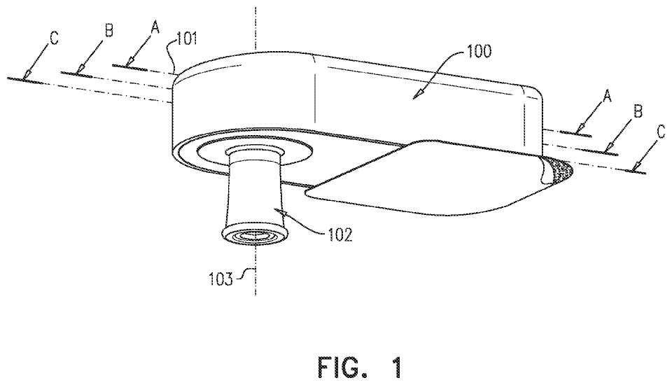

FIG. 22 is a simplified pictorial illustration of a filling port assembly, forming part of the transfusion pump of FIG. 1;

FIGS. 23A and 23B are respective simplified pictorial and sectional exploded view illustrations of the filling port assembly of FIG. 22, FIG. 23B being taken along lines A-A in FIG. 23A;

FIGS. 24A and 24B are respective simplified pictorial and sectional illustrations of a filling port housing, forming part of the filling port assembly of FIGS. 21-23B, FIG. 24B being taken along lines A-A in FIG. 24A;

FIGS. 25A and 25B are respective simplified pictorial and sectional illustrations of a first septum, forming part of the filling port assembly of FIGS. 21-23B, FIG. 25B being taken along lines A-A in FIG. 25A;

FIGS. 26A and 26B are respective simplified pictorial and sectional illustrations of a second septum, forming part of the filling port assembly of FIGS. 21-23B, FIG. 26B being taken along lines A-A in FIG. 26A;

FIGS. 27A and 27B are respective simplified pictorial and sectional illustrations of a conduit element forming part of the filling port assembly of FIGS. 21-23B, FIG. 27B being taken along lines A-A in FIG. 27A;

FIG. 28 is a sectional illustration of a filling port assembly, forming part of the transfusion pump of FIG. 1, FIG. 28 being taken along lines A-A in FIG. 22;

FIGS. 29A, 29B and 29C are respective simplified sectional illustrations of the transfusion pump of FIG. 1 with a filling port assembly of FIGS. 22-28 mounted thereto, taken along respective lines A-A, B-B and C-C in FIG. 1;

FIG. 30 is a simplified pictorial illustration of the transfusion pump of FIG. 1, shown in a first operative orientation;

FIGS. 31A-31C are simplified sectional illustrations of the transfusion pump of FIG. 30 taken along respective lines A-A, B-B and C-C in FIG. 30;

FIG. 32 is a simplified pictorial illustration of the transfusion pump of FIG. 1, shown in a second operative orientation;

FIG. 33 is a simplified pictorial illustration of the transfusion pump of FIG. 1, shown in a third operative orientation;

FIG. 34 is a simplified sectional illustration of the transfusion pump of FIG. 33 taken along lines A-A in FIG. 33; FIG. 35 is a simplified pictorial illustration of the transfusion pump of FIG. 1, shown in a fourth operative orientation;

FIGS. 36A and 36B are simplified sectional illustrations of the transfusion pump of FIG. 35 taken along respective lines A-A and C-C in FIG. 35;

FIG. 37 is a simplified pictorial illustration of the transfusion pump of FIG. 1, shown in a fifth operative orientation;

FIGS. 38A and 389 are simplified sectional illustrations of the transfusion pump of FIG. 37 taken along respective lines A-A and C-C in FIG. 37;

FIG. 39 is a simplified pictorial illustration of the transfusion pump of FIG. 1, shown in a sixth operative orientation;

FIG. 40 is a simplified sectional illustration of the transfusion pump of FIG. 39 taken along lines A-A in FIG. 39;

FIG. 41 is a simplified pictorial illustration of the transfusion pump of FIG. 1, shown in a seventh operative orientation;

FIG. 42 is a simplified sectional illustration of the transfusion pump of FIG. 41 taken along line A-A in FIG. 41;

FIG. 43 is a simplified pictorial illustration of the transfusion pump of FIG. 1, shown in an eighth operative orientation;

FIGS. 44A and 44B are simplified sectional illustrations of the transfusion pump of FIG. 43 taken along respective lines A-A and C-C in FIG. 43;

FIG. 45 is a simplified pictorial illustration of the transfusion pump of FIG. 1, shown in a ninth operative orientation;

FIGS. 46A and 46B are simplified sectional illustrations of the transfusion pump of FIG. 45 taken along respective lines A-A and C-C in FIG. 45;

FIG. 47 is a simplified pictorial illustration of the transfusion pump of FIG. 1, shown in a tenth operative orientation;

FIGS. 48A and 48B are simplified sectional illustrations of the transfusion pump of FIG. 47 taken along respective lines A-A and C-C in FIG. 47;

FIG. 49 is a simplified pictorial illustration of the transfusion pump of FIG. 1, shown in an eleventh operative orientation;

FIGS. 50A-50C are simplified sectional illustrations of the transfusion pump of FIG. 49 taken along respective lines A-A, B-B and C-C in FIG. 49.

### DETAILED DESCRIPTION OF EMBODIMENTS OF INVENTION

Described below in accordance with an embodiment of the present invention is a patch transfusion pump assembly, which is adapted to he secured to a patient body surface and is useful for pumping a medicament through an injection port which is removably mountable onto a patient body surface for defining a medicament injection pathway. The pump assembly generally includes: a timed pumping assembly adapted for pumping the medicament through the injection port at a given time; a removable mounting adapted for removable mounting of the timed pumping assembly onto the injection port, whereby when the timed pumping assembly is mounted onto the injection port, the medicament can be pumped by the time pumping assembly through the medicament injection pathway.

Reference is now made to FIG. 1, which is a simplified pictorial illustration of a transfusion pump with a filling port assembly mounted thereto, constructed and operative in accordance with a preferred embodiment of the present invention.

As seen in FIG. 1, a transfusion pump 100 is generally arranged along a longitudinal axis 101 and a filling port assembly 102 is mounted onto the infusion pump 100 and is arranged along an axis 103, which extends generally perpendicularly to axis 101. It is noted that in an “out of the box” operative orientation, the filling port assembly 102 is removably mounted onto the transfusion pump 100. The transfusion pump 100 is generally also termed as pump assembly.

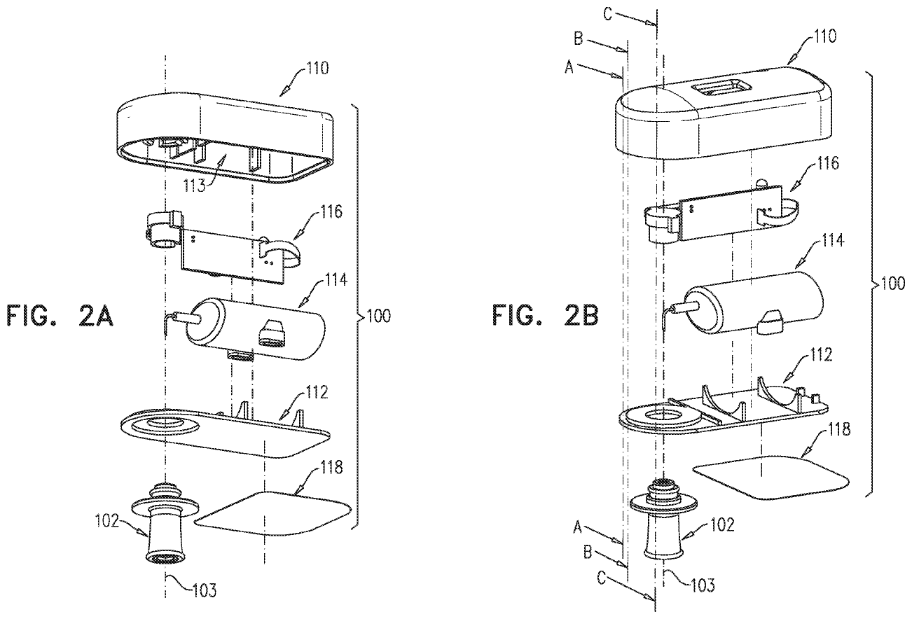

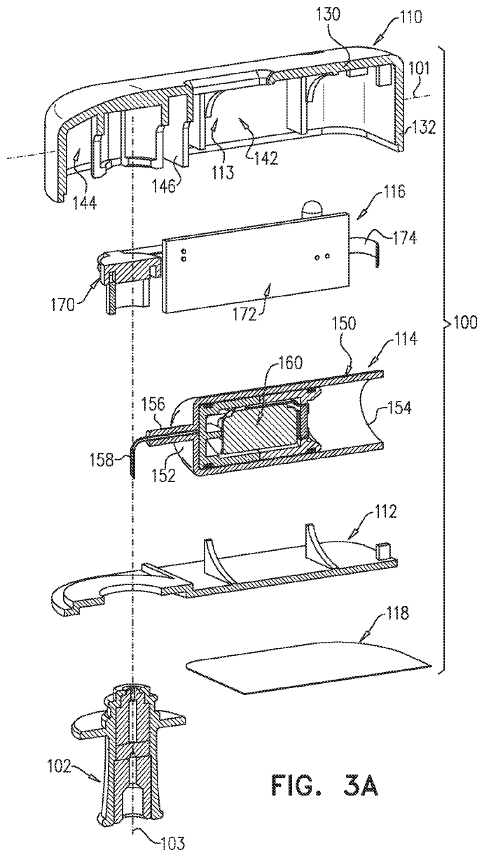

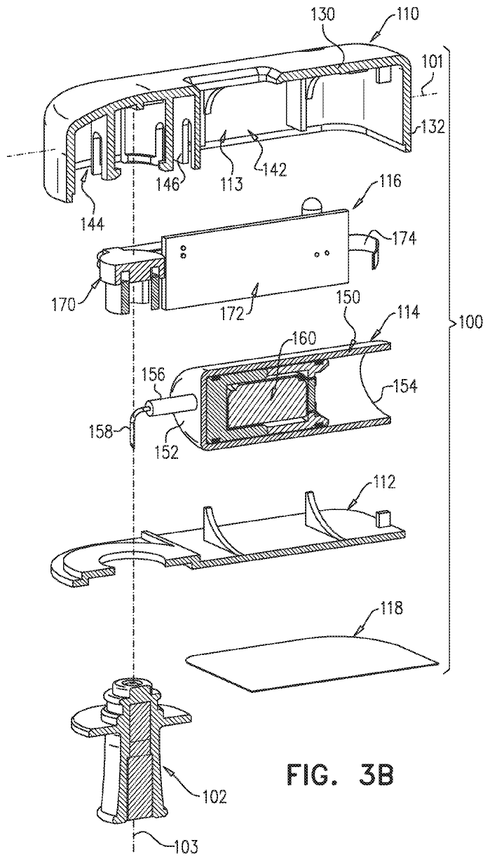

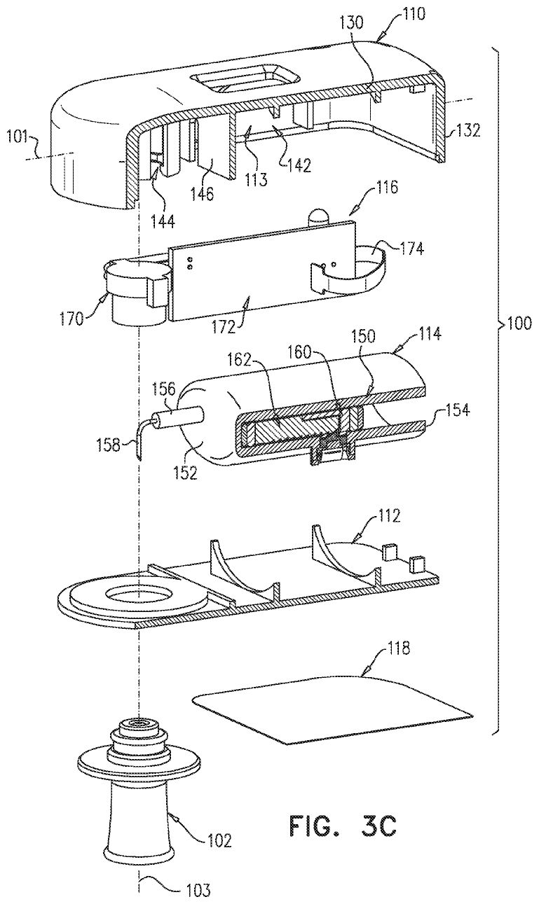

Reference is now made to FIGS. 2A & 2B, which are respective simplified downward-facing and upward-facing exploded view pictorial illustrations of transfusion pump 100 of FIG. 1. Reference is additionally made to FIGS. 3A, 3B and 3C, which are respective simplified exploded view sectional illustrations of the transfusion pump 100 of FIG. 1 taken along respective lines A-A, B-B and C-C in FIG. 2A.

As seen in FIGS. 2A-3C, transfusion pump 100 preferably includes a top housing portion 110, and a bottom housing portion 112 configured to be fixedly mounted to the top housing portion 110 and define an interior volume 113 therebetween, which encloses a reservoir assembly 114 and a circuit and switch assembly 116. An adhesive layer 118 is adapted to at least partially cover the underside of the bottom housing portion 112. The filling port assembly 102 is partially inserted through the bottom housing portion 112 and extends into interior volume 113 formed between the top housing portion 110 and the bottom housing portion 112 of the transfusion pump 100.

it is particularly seen in FIGS. 3A-3C that the top housing portion 110 generally has a flat wall portion 130 and a circumferential wall 132 extending downwardly generally transversely thereto. The outer circumference of the bottom housing portion 112 is suited to fit snugly within the top housing portion 110. The interior volume 113 includes a pump chamber 142 and a fluid delivery and actuation chamber 144. which are separated by a wall 146, which extends downwardly generally transversely with respect to flat wall portion 130.

It is noted that a timed pumping assembly is adapted to reside within the pump chamber 142 and a removable mounting is adapted to reside within the fluid delivery and actuation chamber 144, as described in detail hereinbelow.

The reservoir assembly 114 generally includes a barrel assembly 150 having a forward closed end 152 and a rearward open end 154. The barrel assembly also includes a needle huh 156 extending generally forwardly from closed end 152. A bent needle 158 is partially inserted into the needle hub 156 and is fixedly attached thereto.

It is a particular feature of an embodiment of the present invention that the reservoir assembly 114 encloses a pressure generating assembly 160 and a restraining assembly 162, which is operative to prevent forward displacement of at least a portion of the pressure generating assembly 160 along longitudinal axis 101 until a predetermined pressure level is reached within the pressure generating assembly 160. The restraining assembly 162 is also termed as piston displacement controller.

The circuit and switch assembly 116 includes a switch 170 and a printed circuit board 172. Printed circuit board 172 has electrical contacts 174, which are preferably operatively coupled with at least a portion of the pressure generating assembly 160.

The timed pumping assembly includes at least the reservoir assembly 114 and the circuit and switch assembly 116.

The filling port assembly 102 is partially inserted through the bottom housing portion 112 and partially extends into the fluid delivery and actuation chamber 144 and includes an opening at the upper end thereof, for insertion of the bent needle 156 therethrough and an opening at the lower end thereof, adapted for insertion of a needle of a pre-filled syringe (not shown), as described in detail hereinbelow.

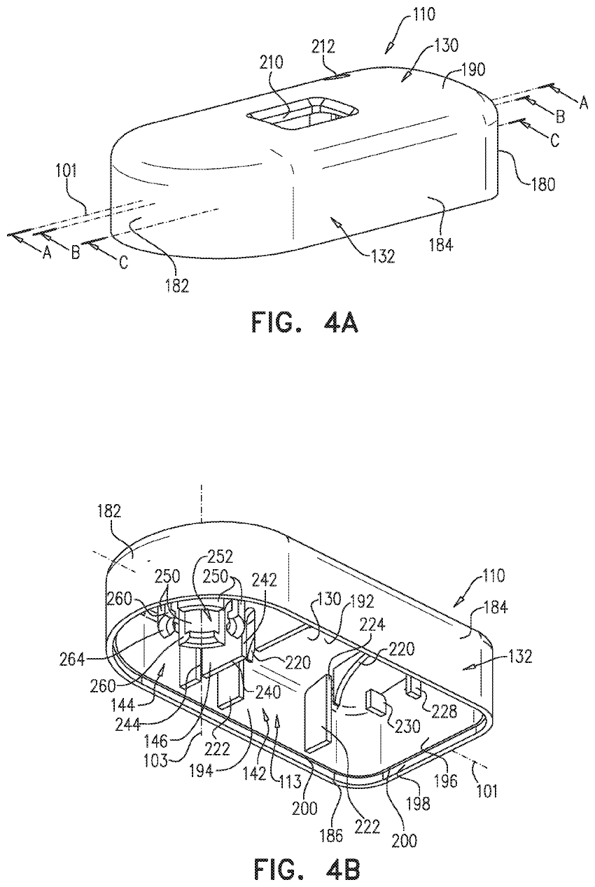

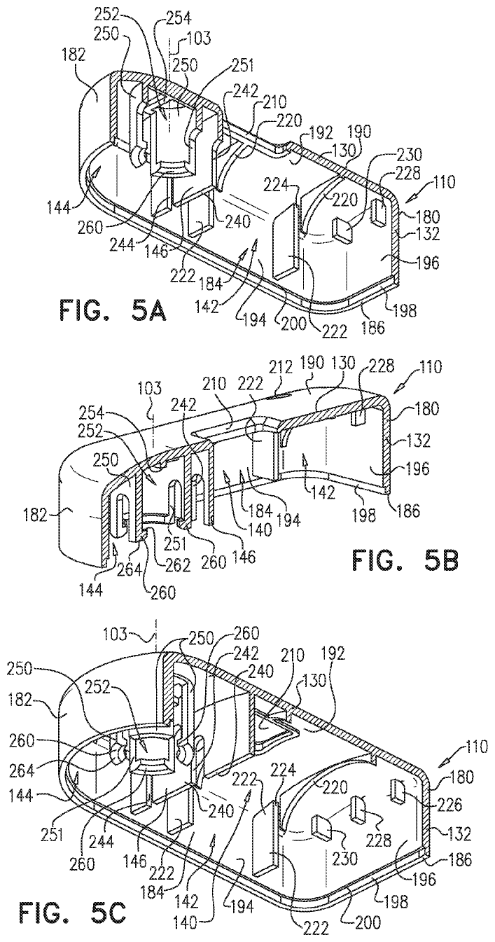

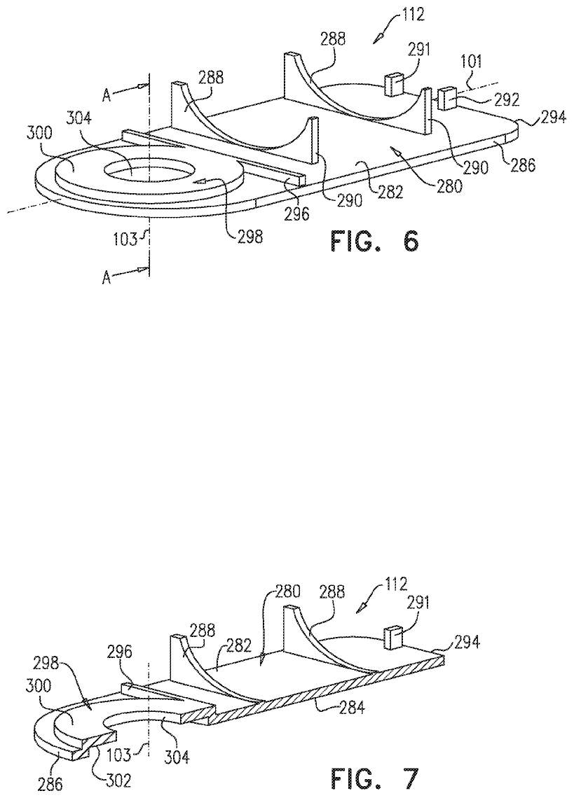

Reference is now made to FIGS. 4A & 4B, which are respective simplified downward-facing and upward-facing pictorial illustrations of the top housing portion 110 of the transfusion pump 100 of FIG. 1. Reference is additionally made to FIGS. 5A, 5B and 5C, which are respective simplified sectional illustrations of the top housing portion 110 of FIGS. 4A & 4B, taken along respective lines A-A, B-B and C-C in FIG. 4A.

The top housing portion 110 is integrally formed from plastic, such as polypropylene and it is generally arranged along longitudinal axis 101.

It is seen in FIGS. 4A & 4B and mentioned hereinabove that top housing portion 110 generally has a flat wall portion 130 and a circumferential wall 132 extending downwardly generally transversely thereto. The interior volume 113 of the top housing portion 110 generally including pump chamber 142 separated from fluid delivery and actuation chamber 144 by separating wall 146, which extends downwardly generally transversely with respect to flat wall 130.

The circumferential wall 132 includes a generally flat rearward wall portion 180, a forward generally arcuate wall portion 182 and two generally spaced side walls 184, each connecting the rearward wall portion 180 with the forward wall portion 182. The circumferential wall 132 defines a downwardly facing circumferential edge 186.

The flat wall portion 130 defines an outer surface 190 and an inner surface 192, the side walls 184 each define an inner surface 194 and the rearward wall portion 180 defines an inner surface 196.

A circumferential recess 198 is formed on the inner surface of the circumferential wall 132 adjacent the circumferential edge 186 and defines a circumferential downwardly facing shoulder 200, which is slightly upwardly spaced from the circumferential edge 186.

A window 210, that is adapted for visual inspection of the contents of the reservoir assembly 114, is preferably formed in flat wall portion 130. Additionally, a LED opening 212, or alternatively, a transparent portion is preferably formed on the top housing portion 110, typically on flat wall portion 130.

It is seen particularly in FIGS. 4B, 5A and 5C that two axially spaced generally arcuate protrusions 220 are formed on flat wall portion 130 and extend generally downwardly from the inner surface 192 thereof The arcuate protrusions 220 are generally spaced apart along longitudinal axis 101 and are adapted for supporting an upward portion of the reservoir assembly 114.

Typically, two ribs 222 extend radially inwardly from the inner surface 194 of one of the side walls 184. The ribs 222 are generally coplanar with arcuate protrusions 220, but forming a gap 224 therebetween, adapted for supporting the circuit and switch assembly 116.

It is also seen in FIGS. 4B-5C that typically three forwardly extending protrusions 226, 228 and 230 are formed on the inner surface 196 of the rearward wall portion 180.

It is further seen that the separating wall 146 defines a downwardly facing edge 240. A recess 242 is formed generally at an intermediate location of the separating wall 146 and extends upwardly from the downwardly facing edge 240 of the separating wall 146. The recess 242 is suited to receive at least a portion of the needle hub 156 therethrough.

A recess 244 is also formed on separating wall 146, extends upwardly from the downwardly facing edge 240 and is laterally spaced from recess 242. Recess 244 is generally coplanar with gap 224 and is preferably adapted for receiving at least a portion of electrical contacts forming part of the circuit and switch assembly 116.

It is also seen in FIGS. 4B-5C that a generally cylindrical protrusion formed of a plurality of mutually concentric arms 250, arranged about axis 103, extends generally downwardly from the inner surface 192 of flat wall portion 130 and disposed within the fluid delivery and actuation chamber 144. The plurality of arms 250 are radially spaced from each other, forming an opening 251 between each pair of arms 250, and defining an inner generally circular volume 252 therewithin.

A protrusion 254 is formed on the inner surface 192 of flat wall portion 130, and disposed within the circular volume 252.

Each of the arms 250 defines a radially inwardly extending snap 260 on its lower end, which has an upwardly facing surface 262 and a downwardly facing surface 264.

It is appreciated that due to the fact that the arms 250 are radially spaced from each other, they are relatively resilient and thus are deflectable upon exertion of radial pressure thereon.

It is noted that the removable mounting includes at least the generally cylindrical protrusion formed of a plurality of mutually concentric arms 250.

Reference is now made to FIGS. 6 & 7, which are respective simplified upward-facing pictorial and sectional illustrations of the bottom housing portion 112 of the transfusion pump 100 of FIG. 1, FIG. 7 being taken along lines A-A in FIG. 6.

The bottom housing portion 112 is integrally formed from plastic, such as polypropylene and is generally arranged along longitudinal axis 101.

It is seen in FIGS. 6 & 7 that bottom housing portion 112 is generally formed of a flat wall portion 280 having an upwardly facing surface 282, a downwardly facing surface 284 and a circumferential edge 286 adapted to fit the corresponding portion of top housing portion 110.

It is seen in FIGS. 6 & 7 that typically two axially spaced generally arcuate protrusions 288 are formed on the flat wall portion 280 and extend generally upwardly from the upwardly facing surface 282 of flat wall portion 280. The arcuate protrusions 288 are generally spaced apart along longitudinal axis 101 and are adapted for supporting a downward portion of the reservoir assembly 114. Each of the arcuate protrusions 288 preferably defines a side edge 290 for supporting the circuit and switch assembly 116.

Typically, two upwardly extending protrusions 291 and 292 are formed adjacent a rearward end 294 of the flat wall portion 280.

A separating rib 296 is formed on the upwardly facing surface 282 of flat wall portion 280 and is disposed forwardly with respect to arcuate protrusions 288. Separating rib 296 extends upwardly from an upwardly facing surface 282 of flat wall portion 280.

A raised annular portion 298 is formed on the upwardly facing surface 282 of flat wall portion 280 and disposed forwardly with respect to separating rib 296. Raised annular portion 298 defines an upwardly facing surface 300 and a downwardly facing annular surface 302, which is slightly upwardly spaced from the downwardly facing surface 284 of the fiat wall portion 280. A bore 304 is formed through the annular portion 298 and is arranged along axis 103. It is noted that bore 304 is suited for partial insertion of the filling port assembly 102 therethrough.

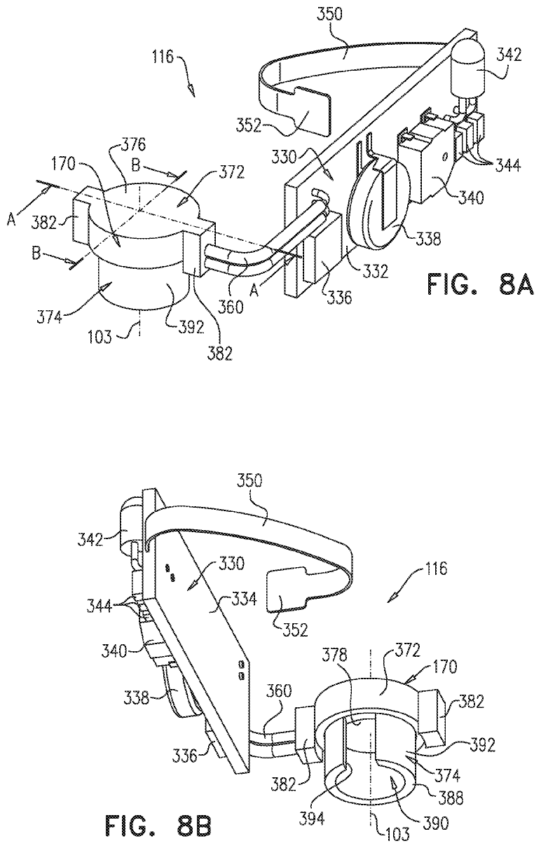

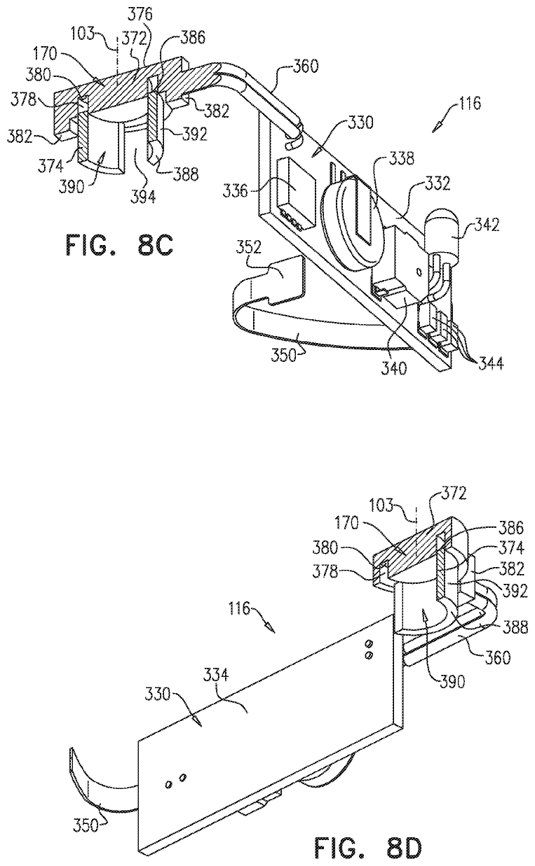

Reference is now made to FIGS. 8A-8D, which are respective simplified first and second pictorial side view illustrations and two sectional illustrations of circuit and switch assembly 116, forming part of the transfusion pump 100 of FIG. 1. sectional illustrations taken along respective lines A-A, B-B in FIG. 8A.

Circuit and switch assembly 116 is made of a rigid printed circuit board 330 defining a first side wall 332 and a second side wall 334. The printed circuit board 330 has various electrical components preferably formed on the first side wall 332, such as a CPU 336, battery 338 adapted to provide electricity to various components of the transfusion pump 100, a buzzer 340 and a LED 342. Various capacitors or resistors 344 may be formed on printed circuit board 330 as well.

A generally flexible electrical cable 350, having electrical contacts 352, extends from one end of the printed circuit board 330 and is operative for electrical coupling with at least a portion of the pressure generating assembly 160, which is enclosed within the reservoir assembly 114.

An electrical cable 360 generally extends from another end of the printed circuit board 330 and is operative for electrical coupling with switch 170.

It is seen in FIGS. 8A-8D that switch 170 is generally arranged along axis 103 and includes an annular portion 372 and a generally cylindrical portion 374, which is partially inserted into the annular portion 372 in an axially slidable manner. The annular portion 372 has an upwardly facing surface 376 and an interior generally annular socket 378 defining a downwardly facing surface 380. Typically, two generally diametrically opposed protrusions 382 are formed on the outer circumference of the annular portion 372 and are adapted to be fitted within a corresponding portion of the top housing portion 110.

The cylindrical portion 374 preferably defines an upwardly facing edge 386, a downwardly facing edge 388, an interior volume 390 and an outwardly facing surface 392. A longitudinal opening 394 is formed in cylindrical portion 374 and extends through the circumferential wall thereof from the upwardly facing edge 386 to the downwardly facing edge 388, being adapted for partial insertion of the needle hub 156 therethrough and into the interior volume 390 of the cylindrical portion 374.

Reference is now made to FIG. 9, which is a simplified pictorial illustration of the reservoir assembly 114. forming part of the transfusion pump 100 of FIG. 1 and to FIG. 10, which is a simplified exploded view illustration of the reservoir assembly 114 of FIGS. 9. Reference is additionally made to FIGS. 11A, 11B and 11C, which are respective simplified sectional illustrations of the reservoir assembly 114 of FIG. 9, taken along respective lines A-A, B-B and C-C in FIG. 10.

The reservoir assembly 114 is seen in FIG. 9. As mentioned hereinabove, the reservoir assembly 114 generally includes barrel assembly 150 having forward closed end 152 and rearward open end 154. Barrel assembly also includes needle hub 156 extending generally forwardly from closed end 152. A bent needle 158 is partially inserted into the needle hub 156 and is fixedly attached thereto. The reservoir assembly 114 is configured to enclose pressure generating assembly 160 and restraining assembly 162, which is operative to prevent forward displacement of at least a portion of the pressure generating assembly 160 along longitudinal axis 101 until a predetermined pressure level is reached within the pressure generating assembly 160.

It is particularly seen in FIGS. 10-11C that the reservoir assembly includes barrel assembly 150 that defines an interior volume 400 having a generally oval cross-section and typically two protrusions 402 extending outwardly from the circumference of the barrel assembly 150. Each of the protrusions 402 defines an interior socket 404, which is configured to receive a ratchet assembly 410 therewithin and communicate with the interior volume 400 of barrel assembly 150.

The interior volume 400 of the barrel assembly 150 preferably encloses a first piston assembly 420, a second piston assembly 430 and a pressure generating element 440, which is configured to be enclosed between the first piston assembly 420 and the second piston assembly 430 at least in some of the operative orientations of the transfusion pump 100. The pressure generating element 440 is also termed as fluid pressure generator.

It is noted that first piston assembly 420, second piston assembly 430 and pressure generating element 440 form at least a portion of the pressure generating assembly 160 and it is particularly seen in FIG. 11B that they are mutually arranged along a longitudinal axis 442 and generally symmetrical thereabout, whereas longitudinal axis 442 is preferably parallel to longitudinal axis 101.

Pressure generating element 440 in accordance with an embodiment of the present invention is a hydrogen cell, such as Cat. Number V150H2MF, commercially available from Varta, Ellwangen, Germany. Alternatively, the pressure generating element 440 can be a compressed gas reservoir or any other suitable pressure generating element.

It is further noted that at least a portion of the first piston assembly 420 and the ratchet assembly 410 preferably forms part of the restraining assembly 162.

It is noted that an alternative restraining assembly 162 can be used in accordance with an embodiment of the present invention. For instance, a valve can be incorporated with bent needle 158, such as a solenoid, which is activated only upon receiving of an electrical signal indicating actuation of pressure generating element 440. Further alternatively, a mechanical valve can be operatively coupled with bent needle 158, which may be activated by pressure exerted thereon following gas discharge from the pressure generating element 440.

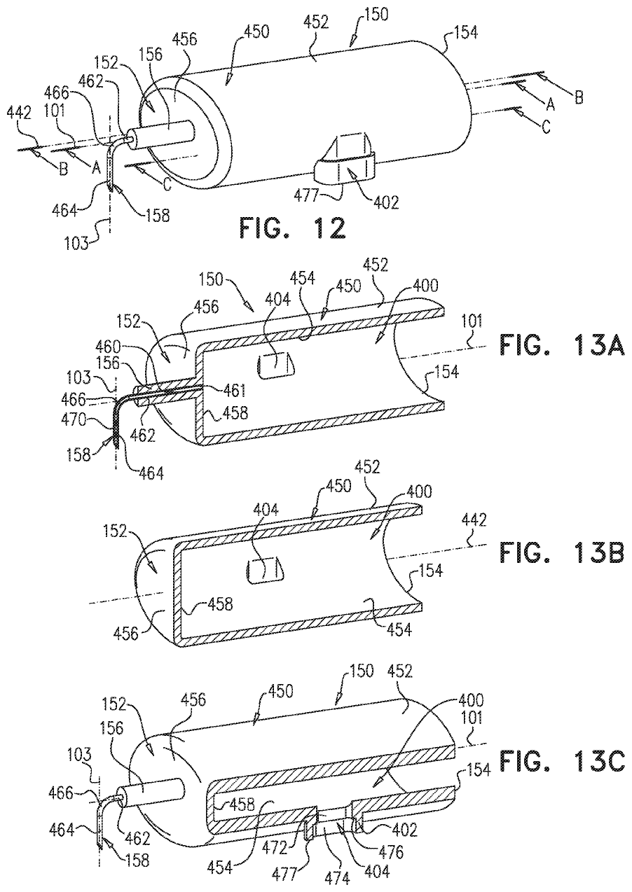

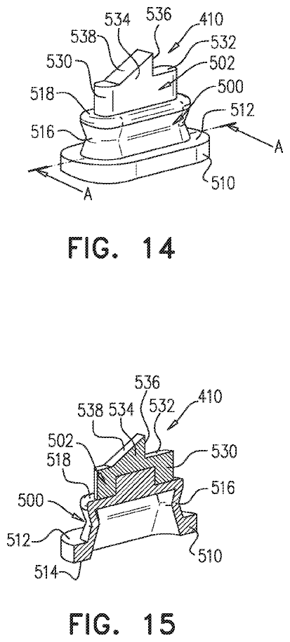

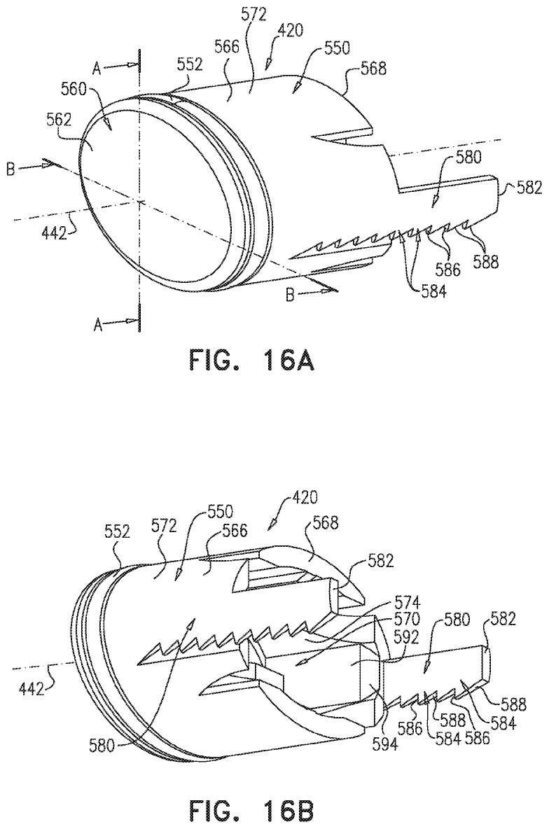

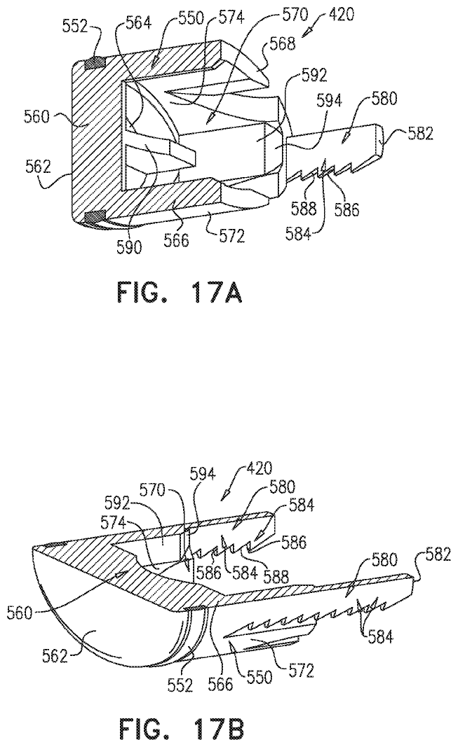

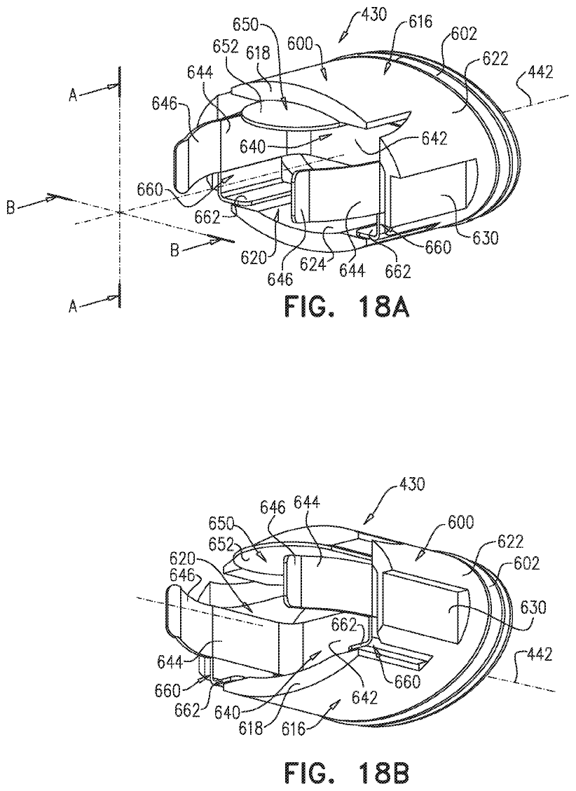

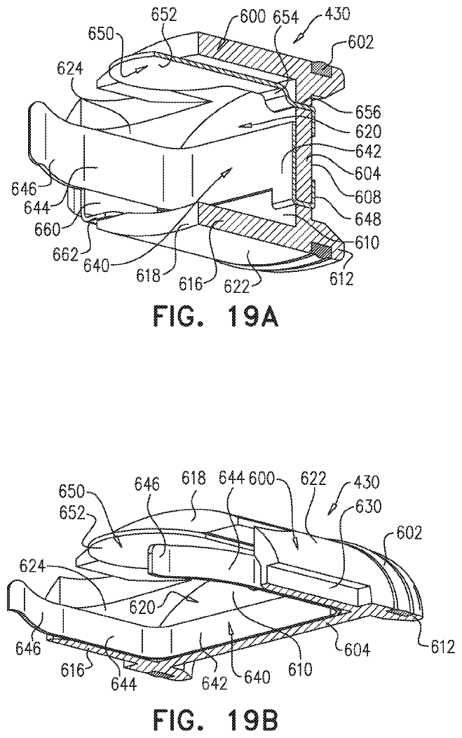

Reference is now made to FIG. 12, which is a simplified pictorial illustration of the barrel assembly 150, forming part of the reservoir assembly 114 of FIGS. 9-12C. Reference is additionally made to FIGS. 13A, 13B and 13C, which are respective simplified sectional illustrations of the barrel assembly 150 of FIG. 12, taken along respective lines A-A, B-B and C-C in FIG. 12.

It is seen in FIGS. 12-13C that the barrel assembly 150 includes an integrally made barrel 450 having a generally oval cross-section, which is preferably made of polypropylene or any other bio-compatible material that doesn't harm the medicament that is adapted to be contained within the interior volume 400 of the barrel assembly 150. The barrel 450 is generally arranged along longitudinal axis 101. Barrel 450 defines an outer surface 452 and an inner surface 454. The forward closed end 152 of the barrel assembly 150 has a forwardly facing surface 456 and a rearwardly facing surface 458.

It is a particular feature of an embodiment of the present invention that the oval cross-section of barrel 450 allows for optimal space utilization within interior volume 113 defined between the top housing portion 110 and bottom housing portion 112 and contributes to pre-defined positioning of the barrel 450 within interior volume 113.

It is further seen in FIGS. 12-13C that generally cylindrical needle hub 156 generally extends forwardly from forward closed end 152 of the barrel assembly 150. The needle hub 156 defines a bore 460 therethrough ending at an aperture 461 formed in the forward closed end 152, which provides for communication between the interior volume 400 of the barrel assembly 150 and the environment through needle 158. Bent needle 158 is partially inserted and fixedly attached within bore 460, by means of adhesive or ultrasonic welding.

It is noted that the bent needle 158 has a first portion 462 extending forwardly from forward closed end 152 of the barrel assembly 150 generally along axis 101 and a second portion 464, which generally extends transversely to the first portion 462 and facing downwardly along axis 103, and a generally arcuate portion 466 connecting therebetween. The bent needle 158 includes a lumen 470 adapted for passage of fluid therethrough.

As noted hereinabove, protrusions 402 are formed on the circumference of the barrel assembly 150 and each of the protrusions 402 define interior socket 404. Each of the sockets 404 has an upper portion 472 having a first cross-section area and a bottom portion 474 having second cross-section area, that is larger than the first cross-section area. A downwardly facing circumferential edge 476 is formed between upper portion 472 and bottom portion 474 and a downwardly facing edge 477 is formed at the bottom end of bottom portion 474.

Reference is now made to FIGS. 14 and 15, which are respective simplified pictorial and sectional illustrations of ratchet assembly 410, forming part of the reservoir assembly 114 of FIGS. 9-11C, FIG. 15 being taken along lines A-A in FIG. 14.

Ratchet assembly 410 preferably includes a generally hollow compressible biasing and sealing element 500 and a ratchet tooth 502, fixedly mounted thereon, preferably by insert molding. The biasing and sealing element 500 is preferably made of a resilient material, such as silicon and ratchet tooth 502 is preferably made of metal, such as copper.

It is seen in FIGS. 14 & 15 that the biasing and sealing element 500 has a generally annular flange 510, which defines an upwardly facing annular shoulder 512 and a downwardly facing surface 514. A compressible bellow portion 516 having a top wall portion 518, which is suited for connection with ratchet tooth 502. The bellow portion 516 is operative to be compressed upon exertion of force on the top wall portion 518 thereof.

Ratchet tooth 502 has a base portion 530, which is mounted onto top wall portion 518 of biasing and sealing element 500. Base portion 530 has an upwardly facing surface 532 with a tooth 534 upwardly extending therefrom. The tooth 534 has a straight rearwardly facing edge 536 and a forwardly tapered edge 538.

Reference is now made to FIGS. 16A and 16B, which are respective simplified first and second side view pictorial illustrations of the first piston assembly 420, forming part of the reservoir assembly 114 of FIGS. 9-11C. Reference is additionally made to FIGS. 17A and 179, which are respective simplified first and second side view sectional illustrations of the first piston assembly 420 of FIGS. 16A & 16B, taken along respective lines A-A and B-B in FIG. 16A.

First piston assembly 420 preferably includes a first piston 550 and a sealing ring 552, which is fixedly mounted thereon. First piston 550 is preferably made of bio-compatible material, such as polypropylene and the sealing ring 552 is preferably made of a relatively resilient material, such as silicon. The first piston 550 has a generally oval cross-section and is generally arranged along axis 442.

The first piston 550 has a generally flat wall portion 560 defining a forwardly facing fluid engagement surface 562 and a rearwardly facing surface 564. A circumferential wall portion 566 extends rearwardly from flat wall portion 560 and defines a rearwardly facing open end surface 568. An interior volume 570 is formed within the first piston 550. Circumferential wall portion 566 defines an outer surface 572 and an inner surface 574.

It is seen in FIGS. 16A-17B that typically two ratchet racks 580 are formed on outer surface 572 of circumferential wall portion 566, each on an opposite side thereof The ratchet racks 580 generally extend longitudinally from a location adjacent to and slightly rearwardly of the sealing ring 552 to a location rearwardly disposed with respect to open end surface 568. Ratchet racks 580 terminate at rearwardly facing edges 582.

Each of the ratchet racks 580 include a plurality of downwardly directed ratchet teeth 584, each defining a straight forwardly facing edge 586 and a rearwardly tapered edge 588.

It is further seen particularly in FIGS. 16B-179 that a generally arcuate protrusion 590 extends rearwardly from rearwardly facing surface 564 of the flat wall portion 560. Arcuate protrusion 590 is suited for supporting the pressure generating element 440. Two generally flat protrusions 592 are formed on the inner surface 574 of circumferential wall portion 566, each on an opposite side thereof, generally aligned with the ratchet racks 580. Rearwardly tapered portions 594 are formed at the rearward ends of protrusions 592, adapted for facilitating insertion of a portion of the second piston assembly 430 into the interior volume 570 of the first piston assembly 420.

Reference is now made to FIGS. 18A and 18B, which are respective simplified first and second side view pictorial illustrations of the second piston assembly 430, forming part of the reservoir assembly 114 of FIGS. 9-11C. Reference is additionally made to FIGS. 19A and 19B, which are respective simplified first and second side view sectional illustrations of the second piston assembly 430 of FIGS. 18A & 18B, taken along respective lines A-A and B-B in FIG. 18A.

Second piston assembly 430 preferably includes a second piston 600 and a sealing ring 602, which is fixedly mounted thereon. Second piston 600 is preferably made of bio-compatible material, such as polypropylene and the sealing ring 602 is preferably made of a relatively resilient material, such as silicon. The second piston 600 has a generally oval cross-section and is generally arranged along axis 442.

The second piston 600 has a generally flat wall portion 604 defining a rearwardly facing surface 608 and a forwardly facing surface 610. A circumferential rim 612 extends slightly rearwardly from flat wall portion 604, it is noted that the sealing ring 602 is disposed at the outer surface of the circumferential rim 612. A circumferential wall portion 616 extends forwardly from flat wall portion 604 and defines a forwardly facing open end surface 618. An interior volume 620 is formed within the second piston 600. Circumferential wall portion 616 defines an outer surface 622 and an inner surface 624.

It is seen in FIGS. 18A-19B that typically two ratchet rack recesses 630 are formed on outer surface 622 of circumferential wall portion 616, each on an opposite side thereof. The ratchet rack recesses 630 generally extend longitudinally from a location adjacent to and slightly forwardly of the sealing ring 602 to the forwardly facing open end surface 618.

It is further seen in FIGS. 18A-19B that a first conductive support portion 640 is fixedly mounted onto flat wall portion 604, preferably by means of insert molding. Support portion 640 is preferably made of metal, such as copper and includes a flat flange portion 642 that abuts the forwardly facing surface 610 and typically two spaced forwardly extending electrically conductive arms 644 adapted to support and operatively electrically communicate with the pressure generator element 440. Each of the forwardly extending arms includes a radially inwardly directed end portion 646. An L-shaped protrusion 648 extends rearwardly from flat flange portion 642 through flat wall portion 604 and configured to fixedly attach the first conductive support portion 640 to second piston 600.

A second conductive support portion 650 is fixedly mounted onto flat wall portion 604, preferably by means of insert molding. Second conductive support portion 650 is preferably made of metal, such as copper and includes a flat electrically conductive flange portion 652 that is disposed above flat flange portion 642 and extends generally forwardly from flat wall portion 604 and is adapted to support and operatively electrically communicate with the pressure generator element 440. Flange portion 652 has a generally curved portion 654 located adjacent the flat wall portion 604 and an L-shaped protrusion 656 extends rearwardly from curved portion 654 through flat wall portion 604 and configured to fixedly attach the second conductive support portion 650 to second piston 600.

Typically, two mutually opposed third support portions 660 are fixedly mounted onto arms 644 of the second piston 600, preferably by means of insert molding. Third support portions 660 are preferably made of metal, such as copper and include a flat flange portion 662 that is disposed below arms 644 and extend radially inwardly, generally transversely with respect to arms 644. Flange portions 662 are adapted to support pressure generating element 440 and enabling proper functioning thereof, as described in detail hereinbelow.

Reference is now made to FIGS. 20A-20E, which are respective simplified forward-facing, rearward-facing and downwardly-facing pictorial illustrations of a partial sub-assembly of the reservoir assembly 114 of FIG. 9 and two sectional illustrations of the partial sub-assembly of the reservoir assembly 114, FIGS. 20D and 20E are taken along respective lines D-D and E-E in FIG. 20A.

It is seen in FIGS. 20A-20E that pressure generating element 440 is held within the second piston assembly 430.

Pressure generating element 440 is of a generally circular cross-section, defining a top surface 680, a bottom surface 682 having at least one opening 684 for facilitating discharge of gas, such as hydrogen therefrom following activation of the pressure generating element 440. Pressure generating element 440 also has a circumferential edge surface 686.

The circumferential edge 686 of the pressure generating element 440 is held by electrically conductive arms 644, the bottom surface 682 of the pressure generating element 440 is held by flange portions 662 and electrically conductive flange portion 652 abuts the top surface 680 of the pressure generating element 440.

It is noted that flange portions 662 are configured to assure that the openings 684 are un-obstructed in any radial orientation of the pressure generating element 440.

Reference is now made to FIGS. 21A, 21B and 21C, which are respective simplified sectional illustrations of the reservoir assembly 114 of FIG. 9, taken along respective lines A-A, B-B and C-C in FIG. 9.

Reservoir assembly 114 is seen in FIGS. 21A-21C in an “out of the box” operative orientation.

It is seen in FIGS. 21A-21C that pressure generating assembly 160 slidably resides within the interior volume 400 of barrel 450, such that sealing rings 552 and 602 of the first piston assembly 420 and the second piston assembly 430 respectively, slidably engage the inner surface 454 of barrel 450. Pressure generating element 440 is enclosed within the interior volumes 570 and 620 defined by the first piston assembly 420 and the second piston assembly 430 respectively and is configured to be electrically coupled to circuit and switch assembly 116 of the transfusion pump 100 through electrical contacts 174 and conductive support portions 640 and 650.

It is seen in FIGS. 21A-21C that a portion of the first piston assembly 420 abuts a portion of the second piston assembly 430, such that forwardly facing open end surface 618 of the second piston assembly 430 abuts rearwardly facing open end surface 568 of the first piston assembly 420. First piston assembly 420 and second piston assembly 430 enclose pressure generating element 440 therebetween, within their respective interior volumes 570 and 620, such that pressure generating element 440 is laterally supported between arcuate protrusion 590 of the first piston assembly 420 and flat flange portion 642 of first conductive support portion 640, which is integrally attached to second piston assembly 430.

It is noted that pressure generating element 440 is operatively electrically coupled to the circuit and switch assembly 116 due to coupling of electrical contact 174 with conductive portions 640, 650 which are fixedly supporting pressure generating element 440.

First piston assembly abuts the forward closed end 152 of barrel assembly 150, such that forwardly facing fluid engagement surface 562 of the first piston assembly 420 abuts the rearwardly facing surface 458 of the forward closed end 152 of the barrel assembly 150.

As seen in FIGS. 21A-21C, outer surface 572 of circumferential wall portion 566 of the first piston assembly 420 and outer surface 622 of circumferential wall portion 616 of the second piston assembly 430 abut the inner surface 454 of the barrel 450. Sealing ring 552 of the first piston assembly 420 and sealing ring 602 of the second piston assembly 430 provide for slidable fluid-tight sealing engagement between each of the piston assemblies 420 and 430 and the inner surface 454 of the barrel 450.

As particularly seen in FIG. 21C, ratchet assembly 410 is disposed within interior socket 404 of the barrel assembly 150 at its non-stressed operative orientation. The compressible bellow portion 516 is seated within bottom portion 474 of the interior socket 404, such that top wall portion 518 of the bellow portion 516 is supported against downwardly facing circumferential edge 476 of the barrel assembly 150. Ratchet tooth 502 is seated within upper portion 472 of the interior socket 404. In this “out of the box” operative orientation, the tooth 534 is disposed rearwardly of the plurality of ratchet teeth 584 of the ratchet rack 580, adjacent the rearwardly facing edge 582 of the ratchet rack 580. Forwardly tapered edge 538 of tooth 534 preferably engages rearwardly tapered edge 588 of one of the teeth 584 of the ratchet rack 580.

Reference is now made to FIG. 22, which is a simplified pictorial illustration of the filling port assembly 102. forming part of the transfusion pump 100 of FIG. 1. Reference is additionally made to FIGS. 23A and 23B, which are respective simplified pictorial and sectional exploded view illustrations of the filling port assembly 102 of FIG. 22, FIG. 23B being taken along lines A-A in FIG. 23A.

It is seen in FIGS. 22-23B that the filling port assembly 102 includes a filling port housing 700 defining an interior volume 702, which is configured for enclosing a first septum 704, a second septum 706 and a conduit element 708. Filling port housing 700, first septum 704, second septum 706 and conduit element 708 are mutually arranged along axis 103.

It is noted above that the filling port assembly 102 is adapted to be removably mounted to the transfusion pump 100.

Reference is now made to FIGS. 24A and 24B, which are respective simplified pictorial and sectional illustrations of the filling port housing 700, forming part of the filling port assembly 102 of FIGS. 21-23B, FIG. 24B being taken along lines A-A in FIG. 24A.

Filling port housing 700 is preferably integrally made of plastic and arranged along axis 103.

As seen in FIGS. 24A and 24B, the filling port housing 700 is a generally hollow element, which preferably includes a gripping portion 710, a mounting portion 712, and a radially outwardly extending annular flange 714 disposed therebetween.

The gripping portion 710 has a generally conical outer surface 720, extending from the annular flange 714 to a slightly outwardly extending circular rim 722.

The mounting portion 712 generally has a circular portion 730 having a first outer diameter, and extending upwardly from the annular flange 714. The first diameter is generally smaller than the outer diameter of the annular flange 714. The circular portion 730 defines an upwardly facing shoulder 732, which is generally disposed in parallel to annular flange 714. A ring portion 734 is formed adjacent to and extending upwardly and in perpendicular to upwardly facing shoulder 732. The ring portion 734 has a second diameter, which is generally smaller than the first diameter. An additional ring portion 736 is disposed adjacent to and above ring portion 734. Ring portion 736 extends generally perpendicularly to ring portion 734 and has a third diameter. which is generally greater than the second dimeter. Ring portion 736 defines a generally circular downwardly facing surface 738 and a circular upwardly facing surface 740. A generally short cylindrical top portion 742 is formed adjacent to and above ring portion 736 and extends generally perpendicularly thereto. The top portion 742 has a fourth diameter, which is generally smaller than the third diameter. Top portion 742 defines an upwardly facing surface 744 with an opening 746 formed therein. The gripping portion 710 defines an opening 748 adjacent circular rim 722.

The interior volume 702 of the filling port housing 700 includes a first portion 750 having a first diameter. First portion 750 extends from opening 748 to a downwardly facing shoulder 752. A second portion 754 having a second diameter, generally smaller than the first diameter, extends from the downwardly facing shoulder 752 to a downwardly facing shoulder 756, disposed generally within ring portion 734. A third portion 758 having a third diameter, generally smaller than the second diameter, extends from the downwardly facing shoulder 756 to downwardly facing shoulder 760, disposed adjacent opening 746. It is noted that in certain operative orientations of the filling port assembly 102, fluid communication is permitted between opening 748 and opening 746.

Reference is now made to FIGS. 25A and 25B, which are respective simplified pictorial and sectional illustrations of the first septum 704, forming part of the filling port assembly 102 of FIGS. 21-23B, FIG. 25B being taken along lines A-A in FIG. 25A.

First septum 704 is preferably integrally made of silicon and arranged along axis 103.

As seen in FIGS. 25A and 25B, first septum 704 includes a first portion 770 having a first diameter, a second portion 772 having a second diameter, generally smaller than the first diameter and extending upwardly therefrom and a tip portion 774 having a third diameter, generally smaller than the second diameter and extending upwardly therefrom. Tip portion 774 is adapted to be pierced by a needle.

The first portion 770 defines a downwardly facing surface 776 and an upwardly facing circular shoulder 778. Second portion 772 extends from shoulder 778 to an upwardly facing shoulder 780. A bore 782 extends upwardly from downwardly facing surface 776, to a location adjacent tip portion 774.

Reference is now made to FIGS. 26A and 26B, which are respective simplified pictorial and sectional illustrations of the second septum 706, forming part of the filling port assembly 102 of FIGS. 21-23B, FIG. 26B being taken along lines A-A in FIG. 26A.

Second septum 706 is preferably integrally made of silicon and arranged along axis 103.

Second septum 706 is a generally circular element having an upwardly facing surface 790 and a downwardly facing surface 792, having a generally tapered notch 794 formed therein.

Reference is now made to FIGS. 27A and 27B, which are respective simplified pictorial and sectional illustrations of the conduit element 708 forming part of the filling port assembly 102 of FIGS. 21-23B, FIG. 27B being taken along lines A-A in FIG. 27A.

Conduit element 708 is preferably integrally made of plastic and arranged along axis 103.

Conduit element 708 is generally cylindrical and has a bore 798 extending therethrough. Bore 798 has a first portion 800 extending from an opening 802 to an aligning opening 804. A tapered surface 806 extends from first portion 800 to opening 804. A second cylindrical bore portion 808 extends from opening 802 to an opening 810 formed on an upwardly facing surface 812 of the conduit element 708.

Reference is now made to FIG. 28, which is a sectional illustration of the filling port assembly 102, forming part of the transfusion pump 100 of FIG. 1, FIG. 28 being taken along lines A-A in FIG. 22.

As seen in FIG. 28, the first septum 704 is sealingly disposed within the interior volume 702 of the filling port housing 700, such that upwardly facing circular shoulder 778 of the first piston 704 abuts downwardly facing shoulder 756 of the filling port housing 700 and upwardly facing shoulder 780 of the first piston 704 abuts downwardly facing shoulder 760 of the filling port housing 700. Tip portion 774 of the first septum 704 protrudes into opening 746 of the tilling port housing 700.

The second septum 706 is sealingly disposed below first septum 704, such that upwardly facing surface 790 of the second septum 706 abuts downwardly facing surface 776 of the first septum 704, thus closing bore 782 formed in the first septum 704 and creating a closed chamber between the first septum 704 and the second septum 706. It is noted that fluid flow through bore 782 is permitted when both septum 704 and 706 are pierced by needles.

The conduit element 708 is seated below the second septum 706, such that upwardly facing surface 812 of the conduit element 708 abuts downwardly facing shoulder 752 of the filling port housing 700 and also abuts downwardly facing surface 792 of the second septum 706. Second cylindrical bore 808 of the conduit element 708 is aligned with notch 794 of the second septum 706.

Reference is now made to FIGS. 29A, 29B and 29C, which are respective simplified sectional illustrations of the transfusion pump 100 of FIG. 1 with the filling port assembly 102 of FIGS. 22 28 mounted thereto, taken along respective lines A-A, B-B and C-C in FIG. 1.

As seen in FIGS. 29A-29C, bottom housing portion 112 is generally attached to top housing portion 110, such that circumferential edge 286 of flat wall portion 280 of bottom housing portion 112 fits within circumferential recess 198 of top housing portion 110 and downwardly facing shoulder 200 of top housing portion 110 overlies upwardly facing surface 282 of bottom housing portion 112. It is also seen that separating rib 296 of bottom housing portion 112 abuts wall 146 of top housing portion 110.

It is further seen that reservoir assembly 114 is fixedly fitted within pump chamber 142 of interior volume 113 formed between the top housing portion 110 and bottom housing portion 112. Outer surface 452 of the barrel 450 of reservoir assembly 114 is supported by arcuate protrusions 220 of top housing portion 110 and by respective arcuate protrusions 288 of bottom housing portion 112. Closed end 152 of the reservoir assembly 114 is supported against wall 146 of the top housing portion 110. Open end 154 of the reservoir assembly 114 is supported against protrusions 226 and 228 of the top housing portion 110 and protrusions 291 and 292 of the bottom housing portion 112 thereby preventing axial displacement of reservoir assembly 114 along axis 442.

It is noted that the circuit and switch assembly 116 is supported by the top housing portion 110, such that the printed circuit board 330 is inserted within gaps 224 of the top housing portion 110 (not shown) and is prevented from axial longitudinal displacement by abutment of printed circuit board 330 with protrusion 230 of the top housing portion 110. Electrical cable 360 of circuit and switch assembly 116 extends through recess 244 of the top housing portion 110 (not shown) into fluid delivery and actuation chamber 144.

As seen in FIGS. 29A-29C, switch 170 is generally disposed within volume 252 formed by arms 250 of the top housing portion 110, such that upwardly facing surface 376 of annular portion 372 of switch 170 is supported against protrusion 254 of the top housing portion 110. Protrusions 382 of the switch 170 are inserted into openings 251 of the top housing portion 110, thereby preventing radial displacement of switch 170.

It is noted that cylindrical portion 374 of switch 170 is selectably slidably attached to annular portion 372 of switch 170, and is configured to be axially slidably displaced along axis 103 upon exertion of force on downwardly facing edge 388 of cylindrical portion 374 and thereby actuate the switch 170. It is appreciated that switch 170 can alternatively be a proximity sensor, magnet switch or a relay switch.

Raised annular portion 298 of bottom housing portion 112 slightly extends into fluid delivery and actuation chamber 144 of top housing portion 110. Needle hub 156 of the reservoir assembly 114 generally extends forwardly through recess 242 formed in wall 146, further through one of the openings 251 formed between arms 250 and still further through opening 394 formed in cylindrical portion 374, such that bent needle 158 extends within interior volume 390 of switch 170.

It is further seen that filling port assembly 102 is removably mounted onto transfusion pump 100, such that annular flange 714 of filling port housing 700 of filling port assembly 102 abuts downwardly facing annular surface 302 of the bottom housing portion 112, such that bottom surface of annular flange 714 is generally flush with downwardly facing surface 284 of the bottom housing portion 112. It is seen that mounting portion 712 of filling port housing 700 extends into fluid delivery and actuation chamber 144 of the top housing portion 110 and is removably secured therein by means of engagement of mounting portion 712 with inwardly extending snaps 260 of arms 250 of the top housing portion 110. Particularly, it is seen in FIG. 29B that inwardly extending snaps 260 of top housing portion 110 are snapped between downwardly facing surface 738 and upwardly facing shoulder 732 of the filling port housing 700.

Once the filling port assembly 102 is mounted onto the transfusion pump 100, second portion 464 of bent needle 158 pierces tip portion 774 of the first septum 704 of the filling port assembly 102 and extends into bore 782 of the first septum 704.

It is noted that first piston assembly 420, second piston assembly 430 and pressure generating element 440 are disposed within interior volume 400 of the reservoir assembly 114 and ratchet assembly 410 is disposed within the interior socket 404 formed within protrusion 402 of barrel 450, as described in detail hereinabove with reference to FIGS. 21A-21C.

It is a particular feature of an embodiment of the present invention that ratchet assembly 410 is operative both for restraining forward displacement of at least a portion of the pressure generating assembly 160 in certain operative orientations and for fluid tight sealing of gas that is generated by the pressure generating element 440 in certain operative orientations. It is particularly seen in FIG. 29C that annular flange 510 of biasing and sealing element 500 of ratchet assembly 410 is securely and sealingly compressed between protrusion 402 of the barrel 450 and flat wall portion 280 of the bottom housing portion 112. Particularly, upwardly facing annular shoulder 512 of annular flange 510 abuts downwardly facing surface 477 of the protrusion 402 and downwardly facing surface 514 of annular flange 510 abuts upwardly facing surface 282 of flat wall portion 280.

Reference is now made to FIG. 30, which is a simplified pictorial illustration of the transfusion pump 100 of FIG. 1, shown in a first operative orientation and to FIGS. 31A-31C, which are simplified sectional illustrations of the transfusion pump 100 of FIG. 30 taken along respective lines A-A, B-B and C-C in FIG. 30.

As seen in FIGS. 30-31C, the transfusion pump 100 is shown in a first operative orientation, being a preparatory operative orientation before filling a medicament into the transfusion pump 100. In this orientation, the transfusion pump 100 is turned upside down in comparison with the orientation illustrated and described with reference to FIGS. 1-29C. In this first operative orientation, the transfusion pump 100 is preferably disposed on outer surface 190 of the top housing portion 110, such that downwardly facing surface 284 of the bottom housing portion 112 and the adhesive layer 118 now face upwardly. The filling port assembly 102 is mounted onto transfusion pump 100 in this operative orientation and extends generally upwardly therefrom along axis 103.

It is noted that spatial relationships between the various elements of the transfusion pump 100 and of the filling port assembly 102 described with reference to FIGS. 21A-21C as well as with reference to FIGS. 29A-29C generally remain unchanged in this first operative orientation.

Specifically, the following spatial relationship characterize the first operative orientation of the transfusion pump 100:

The height of the mounting portion 712 of the filling port housing 700 is not sufficient in order to exert pressure on cylindrical portion 374 of switch 170, thus upwardly facing surface 740 of filling port housing 700 generally abuts downwardly facing edge 388 of cylindrical portion 374, but does not displace it axially along axis 103. It is particularly seen that in this first operative orientation, cylindrical portion 374 is axially spaced from annular portion 372, in such a manner that there is a gap between upwardly facing edge 386 of cylindrical portion 374 and downwardly facing surface 380 of annular portion 372.

It is a particular feature of an embodiment of the present invention that when this gap exists between upwardly facing edge 386 of cylindrical portion 374 and downwardly facing surface 380 of annular portion 372, the switch 170 is disposed in a non-activated operative orientation.

The filling port assembly 102 is mounted onto the transfusion pump 100, such that second portion 464 of bent needle 158 pierces tip portion 774 of the first septum 704 of the tilling port assembly 102 and extends into bore 782 of the first septum 704.

In this first operative orientation, the tip of needle 158 is enclosed between tip portion 774 of first septum 704 and between the second septum 706. There is no fluid communication through lumen 470 of needle 158 since the tip of the needle 158 is enclosed within bore 782, which is closed by the second septum 706 and the opposite end of the needle 158 is sealed by virtue of engagement with flat wall portion 560 of the first piston assembly 420.

Bore 798 of conduit element 708 of the filling port assembly 102 is generally facing upwardly in this first operative orientation and is ready for receiving a luer of a syringe (not shown).

It is a particular feature of an embodiment of the present invention that the pressure generating assembly 160 is disposed at its forwardmost position with respect to barrel 450 in this first operative orientation, such that the flat wall portion 560 of first piston assembly 420 abuts closed end 152 of barrel assembly 150. Particularly, it is seen that fluid engagement surface 562 of flat wall portion 560 abuts rearwardly facing surface 458 of closed end 152.

It is a further particular feature of an embodiment of the present invention that the interior volume 400 of barrel assembly 150 is entirely empty of fluid before filling thereof, due to the fact that the first piston assembly 420 abuts closed end 152 of barrel assembly 150, thus obviating the need for priming of barrel assembly 150 and avoiding any residual air bubbles that may have otherwise remain within the interior volume 400 of barrel assembly 150 after filling of medicament.

It is a still further particular feature of an embodiment of the present invention that the open rearward end of the first piston assembly 420 abuts the open forward end of the second piston assembly 430, in such a manner that rearwardly facing open end surface 568 of the first piston assembly 420 abuts forwardly facing open end surface 618 of the second piston assembly 430 and pressure generating element 440 is tightly supported between the first piston assembly 420 and the second piston assembly 430, as described in detail with reference to FIGS. 21A-21C.

The circumferential rim 612 of the second piston assembly 430 is forwardly longitudinally spaced from protrusions 228 and 230 of top housing portion 110 and protrusions 291 and 292 of bottom housing portion 112.

Filling port assembly 102 partially extends into fluid delivery and actuation chamber 144 of the top housing portion 110 and is removably secured therein by means of engagement of mounting portion 712 with inwardly extending snaps 260 of arms 250 of the top housing portion 110. Particularly, it is seen in FIG. 31B that inwardly extending snaps 260 of top housing portion 110 are snapped between downwardly facing surface 738 and upwardly facing shoulder 732 of the filling port housing 700.

It is particularly seen in FIG. 31C that the annular flange 510 of biasing and sealing element 500 of ratchet assembly 410 is securely and sealingly compressed between downwardly facing surface 477 of protrusion 402 of the barrel 450 and upwardly facing surface 282 of flat wall portion 280 of the bottom housing portion 112.

It is further seen in FIG. 31C that in this first operative orientation the ratchet assembly 410 is disposed within interior socket 404 of the barrel assembly 150 at its non-stressed operative orientation. The compressible bellow portion 516 is seated within bottom portion 474 of the interior socket 404, such that top wall portion 518 of the bellow portion 516 is supported against downwardly facing circumferential edge 476 of the barrel assembly 150. Ratchet tooth 502 is seated within upper portion 472 of the interior socket 404. In this first operative orientation, the tooth 534 is disposed rearwardly of the plurality of ratchet teeth 584 of the ratchet rack 580, adjacent the rearwardly facing edge 582 of the ratchet rack 580. Forwardly tapered edge 538 of tooth 534 preferably engages rearwardly tapered edge 588 of one of the teeth 584 of the ratchet rack 580.

Reference is now made to FIG. 32, which is a simplified pictorial illustration of the transfusion pump 100 of FIG. 1, shown in a second operative orientation.

As seen in FIG. 32, the transfusion pump 100 is shown in a second operative orientation, being a preparatory operative orientation before engagement of a pre-filled. syringe 900 with the filling port assembly 102, that remains mounted onto the transfusion pump 100. In this orientation, the transfusion pump 100 remains turned upside down in comparison with the orientation illustrated and described with reference to FIGS. 1-29C. In this second operative orientation, the transfusion pump 100 is preferably disposed on outer surface 190 of the top housing portion 110, such that downwardly facing surface 284 of the bottom housing portion 112 and the adhesive layer 118 now face upwardly. The filling port assembly 102 is mounted onto transfusion pump 100 in this operative orientation and extends generally upwardly therefrom along axis 103.

It is seen that the pre-filled syringe 900 is ready to engage the filling port assembly 102 and fill the transfusion pump 100 with a medicament 902 (not shown) contained within the pre-filled syringe 900. Syringe 900 preferably includes a barrel 904, which contains the medicament 902 therewithin. The medicament 902 is confined by means of a piston 906 (not shown), which is preferably mounted to or integrally made with a plunger rod 908, that is partially inserted into the interior volume of barrel 904 through an open rearward end of the barrel 904. The barrel 904 has a luer 910 formed at its forward end and a needle 912 forwardly extends axially therefrom along axis 103.

It is noted that the remaining spatial relationships between the various elements of the transfusion pump 100 and of the filling port assembly 102 described with reference to FIGS. 21A-21C, FIGS. 29A-29C and FIGS. 30-31C generally remain unchanged in this second operative orientation.

Reference is now made to FIG. 33, which is a simplified pictorial illustration of the transfusion pump 100 of FIG. 1, shown in a third operative orientation and to FIG. 34, which is a simplified sectional illustration of the transfusion pump 100 of FIG. 33 taken along lines A-A in FIG. 33.

As seen in FIGS. 33 & 34, the transfusion pump 100 is shown in a third operative orientation, being a syringe engagement operative orientation. In this orientation, the transfusion pump 100 remains turned upside down in comparison with the orientation illustrated and described with reference to FIGS. 1-29C.

It is seen that in this third operative orientation, the pre-filled syringe 900 engages the filling port assembly 102. The luer 910 of syringe 900 is received within bore 798 of conduit element 708, such that the tip of needle 912 of syringe 900 pierces the second septum 706 of the filling port assembly 102 and extends into bore 802 of the first septum 704 in order to enable fluid communication between needle 912 and bent needle 158 of the transfusion pump 100, the tip of which also extends into bore 802.

It is noted that during engagement of the syringe 900 with filling port assembly 102, the needle 912 passes through tapered surface 806 of conduit element 708 and tapered notch 794 of second septum 706, both of which facilitate correct alignment and centering of needle 912.

It is noted that the remaining spatial relationships between the various elements of the transfusion pump 100 and of the filling port assembly 102 described with reference to FIGS. 21A-21C, FIGS. 29A-29C and FIGS. 30-31C generally remain unchanged in this third operative orientation.

Specifically, the following spatial relationship characterize the third operative orientation of the transfusion pump 100:

It is further seen that flat wall portion 560 of the first piston assembly 420 engages the closed end 152 of the barrel 450 in the third operative orientation, such that fluid engagement surface 562 of flat wall portion 560 abuts rearwardly facing surface 458 of closed end 152.

The circumferential rim 612 of the second piston assembly 430 is forwardly longitudinally spaced from protrusions 228 and 230 of top housing portion 110 and protrusions 291 and 292 of bottom housing portion 112.

Reference is now made to FIG. 35, which is a simplified pictorial illustration of the transfusion pump 100 of FIG. 1, shown in a fourth operative orientation and to FIGS. 36A and 36B, which are simplified sectional illustrations of the transfusion pump 100 of FIG. 35 taken along respective lines A-A and C-C in FIG. 35.

As seen in FIGS. 35-36B, the transfusion pump 100 is shown in a fourth operative orientation, being an intermediate filling operative orientation. In this orientation, the transfusion pump 100 remains turned upside down in comparison with the orientation illustrated and described with reference to FIGS. 1-29C.

It is seen that in this fourth operative orientation, the patient presses the plunger rod 908 generally downwardly and advances the piston 906 within the barrel 904, such that at least a portion of the medicament 902 contained in the barrel 904 is transferred into the interior volume of the barrel 450 and thus the transfusion pump 100 is partially filled.

Due to exertion of pressure on piston 906 of syringe 900, fluid communication is now produced between the syringe 900 and the transfusion pump 100.

As seen particularly in FIG. 36A, the medicament 902 generally flows from barrel 904 to needle 912 and via bore 782 of the first septum 704 into lumen 470 of the bent needle 158 up to aperture 461 formed in closed end 152 of the barrel 450.

It is a particular feature of an embodiment of the present invention that hydraulic pressure exerted on pressure generating assembly 160 urges rearward sealingly slidable displacement of the pressure generating assembly 160 along longitudinal axis 442 relative to barrel assembly 150. It is specifically seen in FIG. 36A, in the fourth operative orientation, as compared with FIG. 34, which illustrates the third operative orientation, that forwardly facing fluid engagement surface 562 of flat wall portion 560 of the first piston assembly 420 is now partially retracted and rearwardly spaced from rearwardly facing surface 458 of closed end 152 of the barrel assembly 150. The rearward displacement of the pressure generating assembly 160 produces a fluid volume 920 within the reservoir assembly 114. Fluid volume 920 is delimited by rearwardly facing surface 458 of closed end 152 of the barrel assembly 150, inner surface 454 of the barrel 450 and forwardly facing fluid engagement surface 562 of flat wall portion 560.

It is noted that the medicament 902 is sealed within fluid volume 920 due to fluid-tight sealing engagement of sealing ring 552 of the first piston assembly 420 with the inner surface 454 of the barrel 450.

As seen in FIG. 36A, in the fourth operative orientation, as compared with FIG. 34, which illustrates the third operative orientation, the circumferential rim 612 of the second piston assembly 430 is less forwardly longitudinally spaced from protrusions 228 and 230 of top housing portion 110 and protrusions 291 and 292 of bottom housing portion 112.

As seen specifically in FIG. 36B, the ratchet assembly 410 is still disposed within interior socket 404 of the barrel assembly 150 at its non-stressed operative orientation, as shown in FIG. 31C that illustrates the first operative orientation, thus tooth 534 of ratchet assembly 410 engages one of the plurality of ratchet teeth 584 of ratchet rack 580.

As further seen in FIG. 36B, in the fourth operative orientation, as compared with FIG. 31C, which illustrates the first operative orientation, the tooth 534 of the ratchet assembly 410 is disposed at an intermediate location with respect to the plurality of ratchet teeth 584 of the ratchet rack 580 of the first piston assembly 420, and is generally forwardly spaced from the rearwardly facing edge 582 of the ratchet rack 580. Forwardly tapered edge 538 of tooth 534 preferably engages rearwardly tapered edge 588 of one of the intermediate teeth 584 of the ratchet rack 580. Rearward displacement of the pressure generating assembly 160 relative to the barrel assembly 150 is permitted due to the fact that the tapered edge 538 of tooth 534 engages rearwardly tapered edge 588 of any successive tooth of the plurality of teeth 584 of ratchet rack 580 during rearward displacement of the pressure generating assembly 160.

It is a particular feature of an embodiment of the present invention that forward displacement of the pressure generating assembly 160 is prevented by means of engagement of the tooth 534 of ratchet assembly 410 with one of the plurality of teeth 584 of the ratchet rack 580 of the first piston assembly 420 when the compressible bellow portion 516 is disposed in its non-stressed operative orientation. Specifically, forward displacement of the pressure generating assembly 160 is prevented due to engagement of rearwardly facing edge 536 of tooth 534 with forwardly facing edge 586 of one of the plurality of teeth 584 of ratchet rack 580. The engagement of tooth 534 with one of the teeth 584 is urged by the biasing force of compressible bellow portion 516, which is disposed in its non-stressed orientation in this fourth operative orientation of the transfusion pump 100.

It is noted that engagement of the tooth 534 of ratchet assembly 410 with one of the plurality of teeth 584 of the ratchet rack 580 of the first piston assembly 420 serves as a locking mechanism for the first piston assembly 420.

It is a particular feature of an embodiment of the present invention that the switch 170 is disposed in its non-activated orientation in this fourth operative orientation, thus electronically precluding signal transfer indicating initiation of injection of medicament 902 from barrel 450.

It is noted that the remaining spatial relationships between the various elements of the transfusion pump 100 and of the filling port assembly 102 described with reference to FIGS. 33 & 34 generally remain unchanged in this fourth operative orientation.

Reference is now made to FIG. 37, which is a simplified pictorial illustration of the transfusion pump 100 of FIG. 1, shown in a fifth operative orientation and to FIGS. 38A and 38B, which are simplified sectional illustrations of the transfusion pump 100 of FIG. 37 taken along respective lines A-A and C-C in FIG. 37.

As seen in FIGS. 37-38B, the transfusion pump 100 is shown in a fifth operative orientation, being a filled operative orientation. In this orientation, the transfusion pump 100 remains turned upside down in comparison with the orientation illustrated and described with reference to FIGS. 1-29C.

It is seen that in this fifth operative orientation, the patient presses the plunger rod 908 generally downwardly and advances the piston 906 further within the barrel 904, such that preferably the entire amount of the medicament 902 contained in the barrel 904 is transferred into the fluid volume 920 within barrel 450 and thus the transfusion pump 100 is filled.

Due to further exertion of pressure on piston 906 of syringe 900, fluid communication is maintained between the syringe 900 and the transfusion pump 100.

As seen particularly in FIG. 38A, the entire amount of medicament 902 contained in barrel 904 flows through needle 912 and via bore 782 of the first septum 704 into lumen 470 of the bent needle 158 up to aperture 461 formed in closed end 152 of the barrel 450. It is seen that the syringe 900 is empty in this fifth operative orientation and the piston 906 abuts the forward end of the barrel 904.

The hydraulic pressure exerted on pressure generating assembly 160 urges further rearward sealingly slidable displacement of the pressure generating assembly 160 along longitudinal axis 442 relative to barrel assembly 150. It is specifically seen in FIG. 38A, in the fifth operative orientation, as compared with FIG. 36A, which illustrates the fourth operative orientation, that forwardly facing fluid engagement surface 562 of flat wall portion 560 of the first piston assembly 420 is now further retracted and more rearwardly spaced from rearwardly facing surface 458 of closed end 152 of the barrel assembly 150. Further rearward displacement of the pressure generating assembly 160 enlarges fluid volume 920. The pressure generating assembly 160 is now disposed in its rearwardmost position.

It is a particular feature of an embodiment of the present invention that the fluid volume 920 of barrel 450 is now entirely filled with medicament 902, containing no air bubbles, since the interior volume 400 of the barrel assembly 150 before filling was entirely empty of fluid.

As seen in FIG. 38A, in the fifth operative orientation, as compared with FIG. 36A, which illustrates the fourth operative orientation, the circumferential rim 612 of the second piston assembly 430 now abuts protrusions 228 and 230 of top housing portion 110 and protrusions 291 and 292 of bottom housing portion 112, such that further rearward displacement of the pressure generating assembly 160 is prevented.

It is noted that this abutment of circumferential rim 612 with protrusions 228, 230, 291 and 292 defines the rearwardmost position of the pressure generating assembly 160 within reservoir assembly 114 and thus the maximal amount of medicament 902 that can be contained within transfusion pump 100. In case that the desired amount of medicament 902 is lesser than this maximal amount, the circumferential rim 612 will remain forwardly spaced from protrusions 228, 230, 291 and 292.

As seen specifically in FIG. 38B, the ratchet assembly 410 is still disposed within interior socket 404 of the barrel assembly 150 at its non-stressed operative orientation, as shown in Fig, 36B that illustrates the fourth operative orientation, thus tooth 534 of ratchet assembly 410 engages one of the plurality of ratchet teeth 584 of ratchet rack 580.

As further seen in FIG. 38B, in the fifth operative orientation, as compared with FIG. 36B, which illustrates the fourth operative orientation, the tooth 534 of the ratchet assembly 410 is disposed at a forwardmost location with respect to the plurality of ratchet teeth 584 of the ratchet rack 580 of the first piston assembly 420, and is generally more forwardly spaced from the rearwardly facing edge 582 of the ratchet rack 580. Forwardly tapered edge 538 of tooth 534 preferably engages rearwardly tapered edge 588 of the one of the forward teeth 584 of the ratchet rack 580.

It is a particular feature of an embodiment of the present invention that forward displacement of the pressure generating assembly 160 is prevented by means of engagement of the tooth 534 of ratchet assembly 410 with one of the plurality of teeth 584 of the ratchet rack 580 of the first piston assembly 420. Specifically, forward displacement of the pressure generating assembly 160 is prevented due to engagement of rearwardly facing edge 536 of tooth 534 with forwardly facing edge 586 of one of the plurality of teeth 584 of ratchet rack 580. The engagement of tooth 534 with one of the teeth 584 is urged by the biasing force of compressible bellow portion 516, which is disposed in its non-stressed orientation in this fifth operative orientation of the transfusion pump 100.

It is a particular feature of an embodiment of the present invention that the switch 170 is disposed in its non-activated orientation in this fifth operative orientation, thus electronically precluding signal transfer indicating initiation of injection of medicament 902 from barrel 450.

It is noted that the remaining spatial relationships between the various elements of the transfusion pump 100 and of the filling port assembly 102 described with reference to FIGS. 35-36B generally remain unchanged in this fifth operative orientation.

Reference is now made to FIG. 39, which is a simplified pictorial illustration of the transfusion pump 100 of FIG. 1, shown in a sixth operative orientation and to FIG. 40, which is a simplified sectional illustration of the transfusion pump 100 of FIG. 39 taken along lines A-A in FIG. 39.

As seen in FIGS. 39 & 40, the transfusion pump 100 is shown in a sixth operative orientation, being a filling port assembly detachment operative orientation. In this orientation, the transfusion pump 100 remains turned upside down in comparison with the orientation illustrated and described with reference to FIGS. 1-29C.

It is seen that in this sixth operative orientation, the patient detaches the filling port assembly 102 from transfusion pump 100 by means of grasping the gripping portion 710 of filling port housing 700 of filling port assembly 102 and pulling it axially upwardly, thereby causing outwardly radial deflection of arms 250 of top housing portion 110, resulting in disengagement of inwardly extending snaps 260 from mounting portion 712 of the filling port assembly 102.

As seen specifically in FIG. 40, bent needle 158 of the transfusion pump 100 disengages the first septum 704 of the filling port assembly 102 and is now ready for engagement with an injection port (not shown). The bent needle 158 is preferably enclosed and protected within delivery and actuation chamber 144 of top housing portion 110. The needle 912 of the syringe remains sealed within bore 782 of the self-sealing first septum 704 of the filling port assembly 102.

It is a particular feature of an embodiment of the present invention that the switch 170 is disposed in its non-activated orientation in this sixth operative orientation, thus electronically precluding signal transfer indicating initiation of injection of medicament 902 from barrel 450.

It is noted that the remaining spatial relationships between the various elements of the transfusion pump 100 described with reference to FIGS. 37-38B generally remain unchanged in this sixth operative orientation.

Reference is now made to FIG. 41, which is a simplified pictorial illustration of the transfusion pump 100 of FIG. 1, shown in a seventh operative orientation and to FIG. 42, which is a simplified sectional illustration of the transfusion pump 100 of FIG. 41 taken along line A-A in FIG. 41.

As seen in FIGS. 41 & 42, the transfusion pump 100 is shown in a seventh operative orientation, being a pre-attachment to injection port operative orientation. In this orientation, it is seen that an injection pork 950 is already attached to the skin of the patient and the transfusion pump 100 is ready to be mounted onto the injection port 950.

It is noted that the injection port 950 is such as the registered trademark Cleo 90, commercially available from Smiths Medical, Dublin, Ohio, USA. It is appreciated that any other commercial available injection port may be used in accordance with an embodiment of the present invention.

The injection port 950 includes a generally circular base portion 952, having an adhesive layer 954 attached to the underside thereof and a mounting portion 956, which extends upwardly perpendicularly to base portion 952, generally along axis 103. Mounting portion 956 has a circumferential wall portion 958 and a top wall portion 960 extending generally perpendicularly thereto. Generally circular recess 962 is formed within circumferential wall portion 958. An opening 964 is formed within top wall portion 960. Mounting portion 956 has an interior volume, Which preferably encloses a septum 966 and a cannula hub 968. At least a portion of septum 964 partially protrudes through opening 964 formed in top wall portion 960. A cannula 970 generally extends axially along axis 103 from the cannula hub 968 through base portion 952 and protrudes downwardly therefrom. It is noted that cannula 970 constitutes at least a portion of a medicament injection pathway formed between the transfusion pump 100 and the patient through injection port 950.

As seen in FIGS. 41 & 42, the injection port 950 is preferably attached to the skin of the patient in a common manner, by using an inserter device, which enables insertion of the cannula 970 into the injection site. The injection port 950 is secured to the skin of the patient by means of adhesive layer 954 and cannula 970 is inserted into the desired location within the body of the patient, The cannula 970 is sealed from the atmosphere by means of septum 966.

The transfusion pump 100 is turned upside down in comparison with the orientation illustrated and described with reference to FIGS. 30-40 and is now positioned in the orientation illustrated in FIGS. 1-29C.

It is seen that in this seventh operative orientation, the patient holds the transfusion pump 100 pre-mounting thereof to injection port 950 and can visually inspect the medicament 902 contained within fluid volume 920 through window 210 formed on top housing portion 110.

It is a particular feature of an embodiment of the present invention that due to the fact that the injection port 950 is independent of the transfusion pump 100 and that the transfusion pump 100 is configured to be removably mounted onto the injection port 950, the patient or the physician can attach a different injection port 950 to another location on the body surface of the patient, in the event that the location of the first injection port 950 is not convenient for the patient.

It is a particular feature of an embodiment of the present invention that the switch 170 is disposed in its non-activated orientation in this seventh operative orientation, thus electronically precluding signal transfer indicating initiation of injection of medicament 902 from barrel 450.

It is noted that the remaining spatial relationships between the various elements of the transfusion pump 100 described with reference to FIGS. 39-40 generally remain unchanged in this seventh operative orientation.

Reference is now made to FIG. 43, which is a simplified pictorial illustration of the transfusion pump 100 of FIG. 1, shown in an eighth operative orientation and to FIGS. 44A and 44B, which are simplified sectional illustrations of the transfusion pump 100 of FIG. 43 taken along respective lines A-A and C-C in FIG. 43.

As seen in FIGS. 43-44B, the transfusion pump 100 is shown in an eighth operative orientation, being an injection port attachment operative orientation. In this orientation, it is seen that the transfusion pump 100 is mounted onto the injection port 950, which is already disposed at the desired location on the body of the patient.

As seen in FIGS. 43-44B, the transfusion pump 100 is mounted on top of the injection port 950, such that adhesive layer 118 of the transfusion pump 100 adheres the skin of the patient and the mounting portion 956 of the injection port 950 preferably at least partially extends into fluid delivery and actuation chamber 144 of the top housing portion 110 and is removably secured therein by means of engagement of mounting portion 956 with inwardly extending snaps 260 of arms 250 of the top housing portion 110. Particularly, it is seen in FIG. 44A that inwardly extending snaps 260 of top housing portion 110 are snapped within recess 962 of the injection port 950.

At least a portion of the circumferential wall portion 958 of injection port 950 is inserted into bore 304 of bottom housing portion 112 and base portion 952 of the injection port 950 generally abuts downwardly facing annular surface 302 of annular portion 298 of bottom housing portion 112, such that the adhesive layer 954 covering the underside of base portion 952 is generally coplanar with the adhesive layer 118 covering the underside of flat wall portion 280 of the bottom housing portion 112.

The height of the mounting portion 956 of the injection port 950 is sufficient in order to exert pressure on cylindrical portion 374 of switch 170, thus top wall portion 960 of injection port 950 engages downwardly facing edge 388 of switch 170 and slidably displaces cylindrical portion 374 axially upwardly along axis 103. It is seen specifically in FIG. 44A, in the eighth operative orientation, in comparison with FIG. 31A, which illustrates the first operative orientation, that upwardly facing edge 386 of cylindrical portion 374 now abuts downwardly facing surface 380 of annular portion 372.

It is a particular feature of an embodiment of the present invention that when upwardly facing edge 386 of cylindrical portion 374 abuts downwardly facing surface 380 of annular portion 372, the switch 170 is disposed in an activated operative orientation. Assuming activated operative orientation of the switch 170, preferably signals to the CPU 336 of the transfusion pump 100 to initiate a time count to initiation of injection of medicament 902, which is contained within the reservoir assembly 114 of the transfusion pump 100. It is noted that any fixed and pre-determined amount of time may pass between activating switch 170 and initiation of injection, since it may be clinically required to delay injection initiation of medicament 902.

It is noted that the circuit and switch assembly 116 include a timer which is preferably actuated by a time duration start switch, which is being actuated by mounting of the transfusion pump 100 onto the injection port 950.

It is a particular feature of an embodiment of the present invention that the transfusion pump 100 can be removed from the injection port 950 by pulling the transfusion pump 100 axially along axis 103 and thereby causing outwardly radial deflection of arms 250 of top housing portion 110, resulting in disengagement of inwardly extending snaps 260 from recess 962 of the injection port 950.

It is a particular feature of an embodiment of the present invention that due to the fact that the injection port 950 is independent of the transfusion pump 100 and that the transfusion pump 100 is configured to be removably mounted onto the injection port 950. The patient can disconnect the transfusion pump 100 from the injection port 950 at any given time prior to initiation of injection of the medicament 902, and attach a different injection port 950 to another location on the body surface of the patient and then connect the same transfusion pump 100 to the different injection port 950.

It is noted that the circuit and switch assembly 116 stores the elapsed time to initiation of injection within a memory and upon connection of the transfusion pump 100 to a different injection port 950, the timer proceeds counting the remaining time to initiation of injection.

It is a particular feature of an embodiment of the present invention that the circuit and switch assembly 116 continues the time count at the time the transfusion pump 100 is disconnected from the injection port 950 and does not re-set the time count at time of re-connection of transfusion pump 100 to injection port 950.

It is seen specifically in FIG. 44A that once the transfusion pump 100 is mounted onto injection port 950, second portion 464 of bent needle 158 pierces septum 966 of the injection port 950 and thereby provides for fluid flow path between needle 158 of transfusion pump 100 and cannula 970 of injection port 950.

It is a particular feature of an embodiment of the present invention that the pressure generating element 440 is prevented from discharging gas and thereby medicament 902 contained in the reservoir assembly 114 is prevented from entering needle 158 in this operative orientation due to the fact that first piston assembly 420 is prevented from forward displacement until the pre-determined amount of time to initiation of injection is elapsed.

It is a further particular feature of an embodiment of the present invention that the first piston assembly 420 is additionally mechanically prevented from forward axial displacement by means of restraining assembly 162. As specifically seen in FIG. 44B, first piston assembly 420 is prevented from forward displacement by means of engagement of the tooth 534 of ratchet assembly 410 with one of the plurality of teeth 584 of the ratchet rack 580 of the first piston assembly 420. Specifically, forward displacement of the pressure generating assembly 160 is prevented due to engagement of rearwardly facing edge 536 of tooth 534 with forwardly facing edge 586 of one of the plurality of teeth 584 of ratchet rack 580. The engagement of tooth 534 with one of the teeth 584 is urged by the biasing force of compressible bellow portion 516, which is disposed in its non-stressed orientation in this eighth operative orientation of the transfusion pump 100.

It is noted that the restraining assembly 162 prevents the first piston assembly 420 from forward displacement in a medicament pumping displacement direction in this operative orientation.

It is noted that engagement of tooth 534 of the ratchet assembly 410 with one of the teeth 584 of the ratchet rack 580 prevents any discharge of medicament 902 out of barrel 450 until injection initiation, even upon impact on transfusion pump 100.

It is noted that the remaining spatial relationships between the various elements of the transfusion pump 100 described with reference to FIGS. 41 & 42 generally remain unchanged in this eighth operative orientation.

Reference is now made to FIG. 45, which is a simplified pictorial illustration of the transfusion pump 100 of FIG. 1, shown in a ninth operative orientation and to FIGS. 46A and 46B, which are simplified sectional illustrations of the transfusion pump 100 of FIG. 45 taken along respective lines A-A and C-C in FIG. 45.

As seen in FIGS. 45-46B, the transfusion pump 100 is shown in a ninth operative orientation, being an injection initiation operative orientation. In this orientation, the pre-determined time to initiation of injection has elapsed and the pressure generating assembly 440 starts building pressure between the first piston assembly 420 and the second piston assembly 430.

It is a particular feature of an embodiment of the present invention that upon elapsing of the pre-determined amount of time to initiation of injection, and due to electrical connection between circuit and switch assembly 116 and the pressure generating element 440, conducted through first conductive support portion 640 and second conductive support portion 650, pressure generating element 440 is now activated and starts discharging gas and thus building-up pressure between the first piston assembly 420 and the second piston assembly 430. Specifically, pressure generating element 440 discharges Hydrogen through openings 684 formed thereon, thus exerting pressure on ratchet assembly 410 and causing deflection of compressible bellow portion 516. Deflection of compressible bellow portion 516 of the ratchet assembly 410 urges disengagement of tooth 534 from ratchet teeth 584 of ratchet racks 580, thus allowing forward axial displacement of the first piston assembly 420 along axis 442 in order to inject medicament 902 from fluid volume 920 through needle 158 into cannula 970.

As seen specifically in FIG. 46B, upon sufficient pressure build-up between the first piston assembly 420 and the second piston assembly 430, compressible bellow portion 516 of ratchet assembly 410 is deflected and is now disposed in a stressed orientation, thus top wall portion 518 of bellow portion 516 is displaced from downwardly facing circumferential edge 476 of interior sockets 404 and urging disengagement of tooth 534 from ratchet teeth 584.

It is noted that the remaining spatial relationships between the various elements of the transfusion pump 100 described with reference to FIGS. 43-44B generally remain unchanged in this ninth operative orientation.

Reference is now made to FIG. 47, which is a simplified pictorial illustration of the transfusion pump 100 of FIG. 1, shown in a tenth operative orientation and to FIGS. 48A and 48B, which are simplified sectional illustrations of the transfusion pump 100 of FIG. 47 taken along respective lines A-A and C-C in FIG. 47.

As seen in FIGS. 47-48B, the transfusion pump 100 is shown in a tenth operative orientation, being an intermediate injection operative orientation. In this orientation, the first piston assembly 420 separates from the second piston assembly 430 and is displaced axially forwardly along axis 442, thereby affecting injection of medicament 902 from fluid volume 920 into the body of the patient.

It is seen that in this tenth operative orientation that following disengagement of the ratchet assembly 410 from ratchet racks 580 of the first piston assembly 420, the pressure generating element 400 proceeds discharging gas through openings 684 and thereby displaces the first piston assembly 420 axially forwardly along axis 442. Forward displacement of the first piston assembly 420 is now permitted due to the disengagement of ratchet assembly 410 and ratchet racks 580.

It is a particular feature of an embodiment of the present invention that a gas volume 980 is now produced between the first piston assembly 420 and the second piston assembly 430, that is operative for ejecting medicament 902 from fluid volume 920.

Gas volume 980 is preferably delimited by inner surface 574 of the first piston assembly 420, inner surface 454 of the barrel 450, upper portion 472 and bottom portion 474 of interior socket 404 of the barrel 450 and the outer surface of pressure generating element 440.

t is a particular feature of an embodiment of the present invention that the gas contained within gas volume 980 is sealed therewithin at all times due to the fact that annular flange 510 of biasing and sealing element 500 seals the interior socket 404 by means of fluid tight fit of the annular flange 510 between upwardly facing surface 282 of flat wall portion 280 of bottom housing portion 112 and downwardly facing surface 477 of protrusion 402 of barrel 450.

It is seen in FIGS. 48A & 48B that as pressure builds up within the gas volume 980, pressure is exerted on the first piston assembly 420, thereby pushing the first piston assembly 420 further forwardly along axis 442 in a sealingly slidable manner and injects at least a portion of medicament 902 from fluid volume 920 into the lumen 470 of bent needle 158 and further into cannula 970, which delivers the medicament 902 into the desired location within the body of the patient.

It is specifically seen in FIGS. 48A & 48B, in the tenth operative orientation, as compared with FIGS. 46A & 46B, which illustrates the ninth operative orientation, that forwardly facing fluid engagement surface 562 of flat wall portion 560 of the first piston assembly 420 is now partially advanced forwardly and is now less spaced from rearwardly facing surface 458 of closed end 152 of the barrel assembly 150.

It is noted that the medicament 902 is sealed within fluid volume 920 by means of fluid-tight sealing engagement between the inner surface 454 of barrel 450 and the sealing ring 552 of the first piston assembly 420.

As seen particularly in FIG. 48A, in the tenth operative orientation the circumferential rim 612 of the second piston assembly 430 still abuts protrusions 228 and 230 of top housing portion 110 and protrusions 291 and 292 of bottom housing portion 112.

It is noted that it is possible that the transfusion pump 100 was not entirely filled up to its maximal capacity and thus circumferential rim 612 of the second piston assembly 430 was forwardly spaced from protrusions 228 and 230 of top housing portion 110 and protrusions 291 and 292 of bottom housing portion 112. In this case, upon build-up of pressure within gas volume 980, the pressure would initially affect rearward displacement of the second piston assembly 430, up to engagement of the circumferential rim 612 with protrusions 228 and 230 of top housing portion 110 and protrusions 291 and 292 of bottom housing portion 112 and only then pressure within gas volume 980 would affect forward displacement of the first piston assembly 420, thereby axial displacement and ejection of medicament 902 from fluid volume 920.

The contents of the barrel 450 can be inspected by the patient through indication window 210.

It is a particular feature of an embodiment of the present invention that the transfusion pump 100 can be removed from the injection port 950 by pulling the transfusion pump 100 axially along axis 103 and thereby causing outwardly radial deflection of arms 250 of top housing portion 110, resulting in disengagement of inwardly extending snaps 260 from recess 962 of the injection port 950.

It is a particular feature of an embodiment of the present invention that due to the fact that the injection port 950 is independent of the transfusion pump 100 and that the transfusion pump 100 is configured to be removably mounted onto the injection port 950, the patient can disconnect the transfusion pump 100 from the injection port 950 at any given time during injection of the medicament 902 from fluid volume 920 and attach a different injection port 950 to another location and then connect the same transfusion pump 100 with the remaining dosage of medicament 902 to the different injection port 950 and inject the remaining medicament 902.

It is noted that the circuit and switch assembly 116 is pre-programmed such that in the event that the transfusion pump 100 is being disconnected during the injection process, the injection process is stopped. Upon connection of the same transfusion pump 100 to a different injection port 950, the injection of the remaining medicament 902 contained within fluid volume 920 automatically resumes.

It is noted that the remaining spatial relationships between the various elements of the transfusion pump 100 described with reference to FIGS. 45-46B generally remain unchanged in this tenth operative orientation.

Reference is now made to FIG. 49, which is a simplified pictorial illustration of the transfusion pump 100 of FIG. 1, shown in an eleventh operative orientation and to FIGS. 50A-50C, which are simplified sectional illustrations of the transfusion pump 100 of FIG. 49 taken along respective lines A-A, B-B and C-C in FIG. 49.

As seen in FIGS. 49-50B, the transfusion pump 100 is shown in an eleventh operative orientation, being an end of injection operative orientation. In this orientation, the first piston assembly 420 is further displaced axially forwardly along axis 442 under the pressure exerted thereon by gas pressure created within gas volume 980 and is now disposed at its forwardmost position, thereby affecting axial displacement and ejection of the entire amount of medicament 902 from fluid volume 920.

It is seen in this eleventh operative orientation that following disengagement of the ratchet assembly 410 from ratchet racks 580 of the first piston assembly 420, the pressure generating element 400 further proceeds discharging gas through openings 684 and thereby further displaces the first piston assembly 420 axially forwardly along axis 442.

It is seen in FIGS. 50A & 50B that as pressure builds up within the gas volume 980, pressure is exerted on the first piston assembly 420, thereby further pushing the first piston assembly 420 further forwardly along axis 442 in a sealingly slidable manner and injects the entire amount of medicament 902 from fluid volume 920 into the lumen 470 of bent needle 158 and further into cannula 970, which delivers the medicament 902 into the desired location within the body of the patient.

It is specifically seen in FIGS. 50A & 50B, in the eleventh operative orientation, as compared with FIGS. 48A & 48B, which illustrates the tenth operative orientation, that forwardly facing fluid engagement surface 562 of flat wall portion 560 of the first piston assembly 420 now abuts rearwardly facing surface 458 of closed end 152 of the barrel assembly 150, thus assuring that the entire amount of medicament 902 is expelled from fluid volume 920 into the body of the patient.

It is noted that the transfusion pump 100 is preferably adapted to inject a volume of approximately 1 ml of medicament 902 through injection port 950 within a minimal time duration of approximately 5 minutes and the maximal time duration depends on the life-time of the pressure generating element 440.

It is specifically seen in FIG. 50C that due to the pressure build-up within gas volume 980, compressible bellow portion 516 of ratchet assembly 410 remains deflected and disposed in a stressed orientation, thus top wall portion 518 of bellow portion 516 is displaced from downwardly facing circumferential edge 476 of interior sockets 404 and urging disengagement of tooth 534 from ratchet teeth 584.

As further seen in FIG. 50C, in the eleventh operative orientation, as compared with FIG. 48B, which illustrates the tenth operative orientation, the tooth 534 of the ratchet assembly 410 is disposed at the rearwardmost position with respect to the plurality of ratchet teeth 584 of the ratchet rack 580 of the first piston assembly 420, and is generally aligned with the rearwardly facing edge 582 of the ratchet rack 580. Tooth 534 is disengaged from the teeth 584 of the ratchet rack 580.

It is a particular feature of an embodiment of the present invention that the transfusion pump 100 can be removed from the injection port 950 by pulling the transfusion pump 100 axially along axis 103 and thereby causing outwardly radial deflection of arms 250 of top housing portion 110, resulting in disengagement of inwardly extending snaps 260 from recess 962 of the injection port 950.

The transfusion pump 100 is preferably disposable following ejection of the entire amount of medicament 902 from fluid volume 920.

It is noted that the remaining spatial relationships between the various elements of the transfusion pump 100 described with reference to FIGS. 47-48B generally remain unchanged in this eleventh operative orientation.

It is noted that LED 342 may visually indicate to the patient the completion of injection through LED opening 212 formed in top housing portion 110, as specifically seen in FIG. 49. Alternatively, or additionally, indication of injection completion may be provided to the patient audially using buzzer 340 or in a tactile manner, using a vibration motor.

It is a particular feature of an embodiment of the present invention that a piston location sensor is provided within the transfusion pump 100 for sensing a position of the first piston assembly 420 thereby indicating completion of injection of medicament 902. Specifically, a proximity sensor is provided on wall portion 560 of the first piston assembly 420 or on closed end 152 of the barrel 450 in order to identify engagement of forwardly facing fluid engagement surface 562 of wall portion 560 with rearwardly facing surface 458 of closed end 152. Alternatively, an optical sensor can be provided on the inner surface 454 of the barrel 450 or on closed end 152 of the barrel 450 in order to identify engagement of forwardly facing fluid engagement surface 562 of wall portion 560 with rearwardly facing surface 458 of closed end 152. Further alternatively, a magnetic sensor can be provided on the inner surface 454 of the barrel 450 or on closed end 152 of the barrel 450 in order to sense a magnet on wall portion 560 of the first piston assembly 420 in order to identify engagement of forwardly facing fluid engagement surface 562 of wall portion 560 with rearwardly facing surface 458 of closed end 152. Still further alternatively, a switch contactor can be provided either on closed end 152 of the barrel 450 or on wall portion 560 of the first piston assembly 420 adapted to be activated upon engagement of forwardly facing fluid engagement surface 562 of wall portion 560 with rearwardly facing surface 458 of closed end 152.

It is noted that all of the above-mentioned sensors can alternatively identify the maximal distance between wall portion 560 of the first piston assembly 420 and the circumferential rim 612 of the second piston assembly 430, which also indicates completion of injection of medicament 902.

It is a further particular feature of an embodiment of the present invention that a wireless transceiver is provided on circuit and switch assembly 116 of the transfusion pump, which enables wireless data communication between the transfusion pump 100 and a remote communication device, such as a computer or a mobile device.

It is noted that the data that can be wirelessly communicated to a remote communication device preferably includes at least one of the following: indication of injection initiation; indication of injection completion; dosage injected; time of injection; errors or occlusions detected during injection. It is additionally noted, that the transfusion pump 100 may be controlled by the remote communication device, such that a wireless signal is provided by the remote communication device to the wireless transceiver, which enables initiation of injection. Wireless communication may be provided using Bluetooth or any other known wireless protocol.

It is a still further particular feature of an embodiment of the present invention that an environmental sensor is provided on circuit and switch assembly 116 for sensing environmental parameters relevant to timing of injection by the transfusion pump 100. The environmental sensor is adapted for providing an input to a controller provided on the circuit and switch assembly 116. The controller is adapted for providing an operational control input to the transfusion pump based on the input received from the environmental sensor.

The environmental sensor is adapted to identify at least one of the following situations: when the patient is within a high-level radiation area; patient is at a high altitude with low level of oxygen or patient is exposed to a hazardous chemical environment. Upon identifying any of the above-mentioned situations, the environmental sensor provides an input to the controller, which in turn enables automatic initiation of injection or alternatively an adjustment is performed to the pre-determined time to initiation of injection, such that injection can be initiated either before or after the scheduled injection time.

It will be appreciated by persons skilled in the art that the present invention is not limited by what has been particularly shown and described hereinabove. Rather the scope of the present invention includes both combinations and subcombinations of various features described hereinabove as well as variations and modifications thereof which are not in the prior art.

1-75. (canceled)

76. A pump assembly useful for pumping a medicament through an injection port which is removably mountable onto a patient body surface, said pump assembly comprising:
a double piston pumping assembly adapted for pumping said medicament through said injection port, said piston pumping assembly comprising first and second pistons.

77. A pump assembly according to claim 76 and wherein at least one of said first and second pistons moves in a first direction during supply of said medicament to said pumping assembly and said first and second pistons move in mutually operative orientations during pumping of said medicament through said injection port.

78. (canceled)

79. A pump assembly according to claim 76 and wherein said double piston pumping assembly is removably mountable onto said injection port both prior to and following pumping of at least a portion of said medicament through said injection port.

80. A pump assembly according to claim 76 and wherein said double piston pumping assembly is removable from said injection port and attachable to a different injection port without resulting in undesired pumping of said medicament through said injection port.

81-84.(canceled)

85. A pump assembly according to claim 76 and wherein said double piston pumping assembly includes a top housing portion, and a bottom housing portion fixedly mounted to the top housing portion and defining an interior volume therebetween, which encloses a reservoir assembly extending along a longitudinal axis and a circuit and switch assembly, an underside of said bottom housing portion is at least partially covered by an adhesive layer, which is adapted to be secured to said patient body surface.

86. A pump assembly according to claim 85 and wherein said reservoir assembly encloses said first piston, said second piston and a fluid pressure generator, said fluid pressure generator is operatively engaged with at least one of said first and second pistons at least in some of the operative orientations of said double piston pump assembly.

87. A pump assembly according to claim 85 and wherein said first piston has a forwardly facing surface and a rearwardly facing surface and said second piston has a forwardly facing surface and a rearwardly facing surface.

88. A pump assembly according to claim 87, and wherein said forwardly facing surface of said first piston abuts a closed end of said reservoir assembly when said pump assembly is empty of said medicament.

89. A pump assembly according to claim 85 and wherein an interior volume of said reservoir assembly is entirely empty of fluid before filling thereof, thereby obviating the need for priming of said reservoir assembly after filling said reservoir assembly with said medicament.

90. (canceled)

91. A pump assembly according to claim 85 and wherein actuation of said fluid pressure generator produces a gas volume, thereby in turn producing pressure, which is configured to be applied on at least one of said first and second pistons, which pressure is operative for ejecting said medicament from said reservoir assembly.

92-112.(canceled)

113. A pump assembly useful for pumping a medicament through an injection port which is removably mountable onto a patient body surface, said pump assembly comprising:
a piston pumping assembly adapted for pumping said medicament through said injection port, said piston pumping assembly comprising:
a fluid pressure generator;
at least one piston which is driven in a medicament pumping displacement direction by fluid pressure generated by said fluid pressure generator.

114. A pump assembly according to claim 113 and wherein said piston pumping assembly comprising first and second pistons.

115. A pump assembly according to claim 113 and also comprising a piston location sensor for sensing a position of at least one of said at least one piston, thereby indicating completion of injection.

116. A pump assembly according to claim 114 and wherein said fluid pressure generator is operatively engaged with at least one of said first and second pistons at least in some of the operative orientations of said piston pumping assembly.

117. A pump assembly according to claim 114 and wherein at least one of said first and second pistons moves in a first direction during supply of said medicament to said pumping assembly and said first and second pistons move in mutually operative orientations during pumping of said medicament through said injection port.

118. A pump assembly according to claim 113 and wherein said piston pumping assembly is removably mountable onto said injection port both prior to and following pumping of at least a portion of said medicament through said injection port.

119. A pump assembly according to claim 76 and also comprising a piston location sensor for sensing a position of at least one of said first and second pistons, thereby indicating completion of injection.

120. A method of supplying a medicament to a patient comprising the steps of:
removably mounting an injection port onto a body surface of a patient;
thereafter removably mounting a double piston pumping assembly onto said injection port, wherein said piston pumping assembly comprising first and second pistons; and
thereafter operating said pumping assembly to pump said medicament through said injection port.

121. A method of supplying a medicament to a patient according to claim 120 and also comprising a fluid pressure generator, which is operative for driving at least one of said first and second pistons in a medicament pumping displacement direction by fluid pressure generated by said fluid pressure generator.

122. A method of supplying a medicament to a patient according to claim 121 and wherein said double piston piston pumping assembly is removably mountable onto said injection port both prior to and following pumping of at least a portion of said medicament through said injection port.
